(19) United States
(12) Patent Application Publication
(10) Pub. No.: US 2026/0061171 A1
(43) Pub. Date: Mar. 5, 2026

(54) Surgical Wound and Urinary Catheter Drainage Medical Device

(71) Applicant: Maurice Marcel Garcia, Los Angeles, CA (US)

(72) Inventor: Maurice Marcel Garcia, Los Angeles, CA (US)

(21) Appl. No.: 19/378,533

(22) Filed: Nov. 4, 2025

Related U.S. Application Data
(60) Provisional application No. 63/690,925, filed on Sep. 5, 2024.

(51) Int. Cl. A61M 27/00; A61M 39/10; A61M 39/22

(52) U.S. Cl. CPC A61M 27/00; A61M 39/105; A61M 2039/226

(57) ABSTRACT

A surgical drainage system for maintaining tube patency comprises a screw-coil assembly configured to rotate within a thoracostomy tube lumen, a modified thoracostomy tube with dual-lumen configuration, and an automated control system. The screw-coil assembly includes an inner core with helical blades connected by struts, utilizing Archimedes screw principles to transport fluid and clotted material from drainage inlet to outlet through mechanical conveyance. The dual-lumen tube provides dedicated pathways for screw-coil operation and sterile drainage. The automated control system provides programmable intermittent operation to maintain tube patency while avoiding harmful negative pressures generated by conventional manual clearing methods, offering cost-effective automated clearance of thoracostomy tube obstructions.

### CROSS-REFERENCE TO RELATED APPLICATIONS

This application claims the benefit of and priority to U.S. Provisional Patent Application No. 63/690,925, filed Sep. 5, 2024, entitled “Thoracostomy Surgical Wound Drainage Medical Device,” under 35 U.S.C. § 119(e), the entire disclosure of which is hereby incorporated by reference.

### FIELD OF THE INVENTION

The present invention relates to medical drainage systems and, more particularly, to gravity-independent surgical drainage devices configured to maintain tube patency and optimize fluid evacuation from body cavities during post-operative care. The invention encompasses novel drainage tube assemblies with integrated mechanical transport mechanisms, fluid diversion systems, and associated control systems for preventing occlusions in surgical drainage applications including thoracostomy drainage, abdominal drainage, pericardial drainage, and other body cavity drainage procedures where clotting and tube obstruction compromise drainage function.

### BACKGROUND OF THE INVENTION

Surgical drainage tubes are critical medical devices used to evacuate fluid and air from body cavities following surgical procedures. Thoracostomy drainage tubes, representing one important application of surgical drainage technology, must maintain adequate negative pressure within the pleural space to ensure proper lung expansion following cardiac surgery, lung resection, esophageal surgery, and chest trauma procedures, preventing life-threatening complications such as pneumothorax, decreased lung capacity, and cardiac tamponade. Similar drainage challenges exist across multiple surgical contexts including abdominal surgery, pericardial procedures, and other body cavity operations where post-operative fluid accumulation must be effectively evacuated to prevent complications.

A significant challenge across surgical drainage applications is the accumulation of clotted blood within drainage tubes, compromising tube patency and preventing effective fluid evacuation. In thoracostomy drainage specifically, studies indicate that 36% of patients develop completely clogged chest tubes following cardiac surgery, with most occlusions occurring within internal portions of the tube where they cannot be detected by standard bedside assessment. These clot-induced occlusions create inadequate evacuation of blood from the pleural space, leading to retained blood that induces inflammation and fibrosis (fibrothorax) within the pleural space. Similar clotting complications occur across other surgical drainage contexts, where blood accumulation following operative procedures can coagulate within drainage tubes and compromise drainage function.

Current approaches to address tube blockages include manual manipulation techniques such as milking and stripping of the tubing. However, these methods have demonstrated questionable effectiveness and can be dangerous to patients. Stripping as little as 5 cm of tubing can generate mean negative pressures of −87 cm of water, with full stripping reaching dangerous levels of −400 cm of water. Such excessive negative pressure can cause tissue entrapment, increased bleeding, and left ventricular dysfunction.

Active tube clearance (ATC) systems using guidewire-based devices have been developed to address these limitations in thoracostomy applications. While these systems can reduce postoperative complications and associated costs, they suffer from significant practical limitations. Current ATC devices require manual actuation and routine staff maintenance, become nonfunctional in kinked tubing, and are prohibitively expensive, with single-use devices costing approximately $395—more than 25 times the cost of standard chest tubes. These economic and operational limitations have prevented widespread adoption of existing ATC technologies across the broader spectrum of surgical drainage applications.

Existing surgical drainage tube designs present fundamental limitations that compromise both mechanical clearing capability and drainage efficiency. Conventional drainage tubes comprise simple cylindrical structures with drainage holes positioned at the distal end and a single-lumen design that provides no dedicated pathway for mechanical clearance devices while maintaining sterile drainage. The typical configuration features a conical distal end with apertures smaller than the tube diameter, creating a geometry that is incompatible with efficient mechanical clearing systems while limiting drainage flow optimization.

Furthermore, current tube configurations fail to address the fluid dynamics principles that could optimize drainage flow, particularly regarding the directional flow characteristics, helical transport geometry, and connection interface designs that influence fluid transport efficiency. The proximal end connection systems of conventional drainage tubes lack features that would enable integration of mechanical clearing systems while preserving separate sterile drainage pathways, creating a fundamental impediment to development of automated clearing solutions.

The internal geometry of conventional drainage systems lacks features that would naturally promote fluid movement from inlet to outlet independent of gravity-based drainage. Standard tubes provide no mechanism to generate the directional transport forces necessary for gravity-independent fluid evacuation, limiting their effectiveness in applications where patient positioning or anatomical considerations make gravity-assisted drainage suboptimal. This limitation affects not only thoracostomy applications but extends across the full spectrum of surgical drainage contexts including abdominal drains, pericardial drainage systems, and other body cavity drainage applications where effective fluid removal is essential for post-operative recovery.

Additionally, current drainage tube designs fail to adequately address the connection interface requirements necessary for integrating mechanical clearing systems with sterile drainage collection. Conventional single-outlet designs provide no dedicated entry point for mechanical clearing devices while maintaining preserved outflow pathways for sterile drainage collection. This fundamental design limitation prevents effective integration of automated clearing systems that could maintain tube patency throughout the post-operative period without requiring manual intervention.

There remains a need across surgical drainage applications for an automated, cost-effective drainage system that can maintain drainage tube patency without generating excessive pressure changes, while incorporating novel tube designs that enable mechanical clearing integration, optimize fluid flow dynamics through helical transport mechanisms configured for directional fluid movement, and provide connection interfaces that separate mechanical clearing pathways from sterile drainage collection. Such a system should integrate seamlessly with existing clinical workflows while providing economic advantages over current solutions and applicability across multiple surgical drainage contexts beyond thoracostomy applications.

### SUMMARY OF THE INVENTION

In accordance with the invention, the persistent clinical challenge of surgical drainage tube obstruction caused by blood clot accumulation is solved by an integrated gravity-independent drainage system that utilizes the mechanical principles of a directional Archimedes screw to transport fluid and clotted material from the drainage inlet toward the outlet without generating the harmful negative pressures associated with conventional manual clearing methods. The invention provides a comprehensive solution applicable across multiple surgical drainage contexts including thoracostomy drainage, abdominal drainage, pericardial drainage, and other body cavity drainage applications where clotting compromises tube patency.

The inventive drainage system comprises three coordinated components: a directional screw-coil assembly with optimized handedness for distal-to-proximal fluid transport, a novel surgical drainage tube design with integrated connection interface, and an automated control system that work together to maintain tube patency while addressing the safety and economic limitations of existing solutions.

The directional screw-coil assembly represents the core inventive element and incorporates advanced blade geometry and helical handedness specifically optimized for medical drainage applications requiring distal-to-proximal fluid transport. The helical blades feature a concave inner surface configuration on the fluid egress side that creates an enhanced cupping effect, increasing the mechanical force applied to drainage material during rotation, similar to principles observed in swimming propulsion where cupped hand positions generate greater propulsive force than flat orientations. This cupping geometry, combined with a convex outer surface on the fluid inlet side, maximizes the directional flow characteristics fundamental to efficient mechanical transport of both liquid and clotted drainage material.

The helical coil handedness is specifically configured to create fluid transport from the distal end (deepest in the patient where fluid enters the drainage system) toward the proximal end (outside the patient where fluid exits to collection systems) through the relationship between rotational direction and helical thread orientation. In accordance with embodiments, the screw-coil assembly comprises a left-hand helical configuration that, when rotated in a clockwise direction as viewed from the proximal end, creates mechanical transport from distal to proximal, or alternatively comprises a right-hand helical configuration that, when rotated in a counter-clockwise direction as viewed from the proximal end, creates the same distal-to-proximal transport effect. This directional handedness represents a critical design parameter that ensures effective fluid movement independent of gravity assistance.

The structural struts connecting the helical blades to the central rotating bar are configured at optimized angles relative to the central axis to enhance fluid flow dynamics that complement the primary helical transport action. In accordance with embodiments, the struts are oriented at angles less than 90 degrees relative to the central rotating bar, angled toward the distal end of the drainage tube to create flow patterns that favor distal-to-proximal fluid movement. This strut angle optimization represents a departure from conventional perpendicular strut orientations and provides enhanced transport efficiency through improved fluid capture and directional guidance during rotational operation.

The novel surgical drainage tube design incorporates a fundamental departure from conventional drainage tubes through a specialized proximal end configuration that provides a dedicated entry point for the screw-coil assembly while maintaining a separately preserved dedicated outflow lumen for sterile drainage collection. This novel tube design represents a critical inventive element that distinguishes the system from prior art approaches that merely insert clearing devices into conventional drainage tubes. The proximal end configuration enables coaxial integration of the screw-coil mechanism with efficient fluid diversion to a separate collection pathway, fundamentally different from standard conical distal-end drainage tubes with apertures smaller than the tube diameter.

In accordance with embodiments where the screw-coil can be inserted substantially straight into the drainage tube, the drainage may occur through the same aperture where the coil enters at the proximal end, with no benefit to separating the two lumens for fluid flow dynamics within the tube body. In these embodiments, the Y-connector configuration at the proximal end provides fluid diversion where drainage material flowing up the tube is naturally diverted to a wider soft-angled outflow port arranged coaxially with the drainage tube, while the screw-coil insertion port provides dedicated access for the mechanical clearing mechanism. This urinary catheter-style Y-end configuration enables efficient fluid separation at the proximal interface without requiring dual-lumen separation throughout the tube length.

In alternative embodiments, the novel tube design incorporates a dual-lumen configuration throughout the tube length where drainage occurs through a separate aperture in the tube wall itself, maintaining dedicated pathways for screw-coil operation and sterile drainage flow. This dual-lumen embodiment provides complete separation between the mechanical clearing mechanism and the drainage flow pathway, offering advantages in specific clinical scenarios where enhanced sterile isolation is desired.

Multiple safety features address concerns about tissue protection, including protective struts positioned around drainage holes, tapered screw-coil configurations near the tube tip, and precise positioning of the screw-coil assembly relative to drainage openings to prevent tissue damage during clearing operations. A cone-shaped tube tip prevents screw-coil escape while maintaining adequate drainage flow.

The automated control system provides programmable intermittent operation based on characterized clotting patterns, eliminating the need for manual actuation and routine staff maintenance associated with existing active tube clearance systems. The system operates within a controlled speed range of 100 to 20,000 RPM with directional rotation matched to the screw-coil handedness to ensure consistent distal-to-proximal transport, enabling gentle mechanical transport at lower speeds and clot morcellization at higher speeds when enhanced clearing capability is required.

The invention is applicable across multiple surgical drainage contexts beyond thoracostomy applications. The gravity-independent transport mechanism enables effective drainage in abdominal surgical applications, pericardial drainage, and other body cavity drainage scenarios where clotting compromises tube patency and where patient positioning or anatomical factors make gravity-assisted drainage suboptimal. The scalable design accommodates various tube sizes and drainage volumes across different surgical specialties while maintaining the fundamental mechanical transport principles.

By maintaining continuous tube patency through automated mechanical transport with directional flow characteristics, the inventive system enables effective evacuation of blood and fluid from surgical sites, preserving drainage function essential for post-operative recovery and preventing the accumulation of retained blood that induces inflammation and complications. This automated approach addresses the practical limitations of existing solutions while providing significant economic advantages, utilizing cost-effective materials and reducing the ongoing operational costs associated with manual device actuation and maintenance.

The invention thus provides a comprehensive solution that maintains surgical drainage tube function across multiple clinical applications throughout the treatment period, reducing the risk of post-operative complications while offering practical advantages for clinical implementation and patient care.

### BRIEF DESCRIPTION OF THE FIGURES

The accompanying drawings, which are incorporated herein and constitute part of this specification, illustrate various embodiments of the invention and, together with the description, serve to explain the principles of the invention.

FIG. 1 is a perspective view of a thoracostomy drainage system in accordance with an intended use showing the overall system configuration including the Y-connector, motor, housing body, sterile drain chamber, and coils in accordance with an embodiment of the invention.

FIG. 2a is a perspective view showing the coils, central rotating bar, and struts of the screw-coil assembly within the thoracostomy tube lumen in accordance with an embodiment.

FIG. 2b is an alternative perspective view of the screw-coil assembly configuration showing the relationship between the coils, central rotating bar, and struts with directional flow indicators for drainage inlet (distal end) and drainage outlet (proximal end) in accordance with an embodiment.

FIG. 3 is a cross-sectional view illustrating the dual-lumen configuration of the modified thoracostomy tube showing the housing body, central rotating bar, side tubes, and two-lumen separator in accordance with an embodiment.

FIG. 4a is a cross-sectional view showing the external tube attachment system with the Y-connector, motor, side tubes, two-lumen separator, and protective struts in accordance with safety embodiments of the invention.

FIG. 4b is a cross-sectional view showing the internal tube attachment system and the relationship between the two-lumen separator and side tubes within the dual-lumen thoracostomy tube configuration in accordance with an embodiment.

FIG. 5 is a cross-sectional view showing the integration of the motor, housing body, sterile drain chamber, coils, fluid flow pathways, and side tubes in accordance with an embodiment.

FIG. 6a is a cross-sectional view depicting the housing body, sterile drain chamber, coils, fluid, and tissue with protective configurations at the commencement of drainage activities in accordance with embodiments.

FIG. 6b is a cross-sectional view showing the motor, housing body, coils, fluid, central rotating bar, and side tubes following commencement of drainage activities in accordance with an embodiment.

FIG. 7a is a perspective view showing the motor housing, Y-connector, housing body, sterile drain chamber, coils, and animal (in this case, a pig) utilization demonstrating device validation testing in accordance with an intended non-human use embodiment of the invention.

FIG. 7b is a perspective view illustrating assorted sizes configurations and alternative embodiments of the thoracostomy drainage system with directional flow indicators showing drainage inlet (distal end) and drainage outlet (proximal end) in accordance with various embodiments.

### DETAILED DESCRIPTION

In accordance with an embodiment, the present invention addresses the significant clinical challenge of thoracostomy drainage tube obstruction caused by blood clot accumulation. As described in the Background, conventional approaches to maintaining tube patency suffer from limited effectiveness and potential safety concerns, creating a need for an automated, cost-effective solution that can reliably clear obstructions without generating harmful pressure changes.

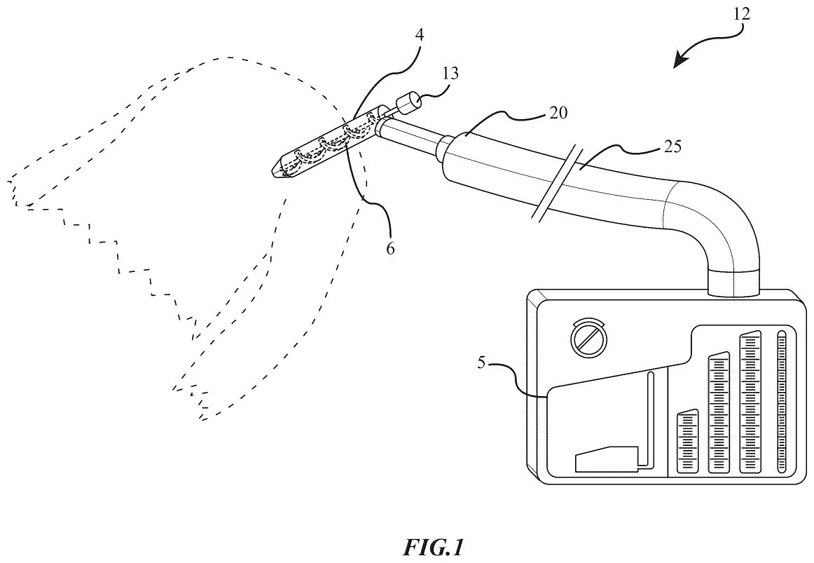

In accordance with an embodiment of the invention, FIG. 1 illustrates a perspective view of the complete thoracostomy drainage system showing the overall system configuration and component integration. The overall concept (12) demonstrates the assembled system architecture integrating mechanical clearing capability with sterile drainage collection functionality. The housing body (4) provides the outer structural envelope of the thoracostomy tube assembly, configured to accommodate both the mechanical clearing system and the essential drainage function while maintaining biocompatible interfaces with patient tissue. The motor housing (13) encases and protects the motor and associated control electronics while providing a sterile interface between the mechanical actuation system and the thoracostomy tube assembly. The side tubes (20) represent the separate drainage pathways within the dual-lumen configuration, specifically dimensioned and positioned to provide adequate cross-sectional area for efficient drainage flow while maintaining separation from the mechanical screw-coil operation. The drainage tube (25) comprises the external thoracostomy tube structure that interfaces with the patient's pleural space and provides the primary conduit for fluid evacuation from the drainage site. The coils (6) represent the helical blade components of the screw-coil assembly that create the mechanical conveyor effect for transporting clotted material through Archimedes screw principles. The sterile drain chamber (5) provides a contained environment for collecting transported drainage material, maintaining sterile conditions while accommodating the fluid and clotted material that is mechanically conveyed by the screw-coil assembly from the thoracostomy tube inlet region.

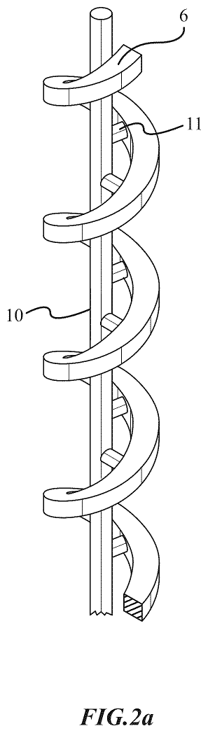

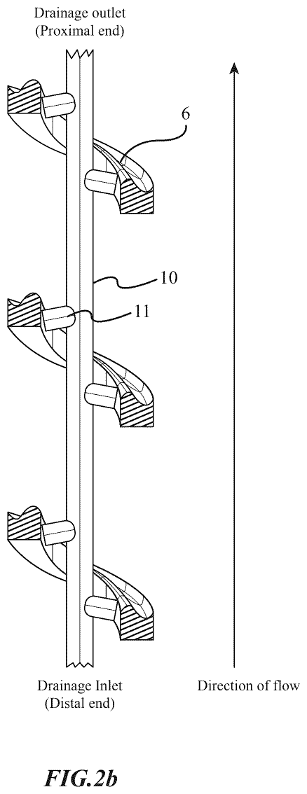

In accordance with an embodiment of the invention, FIG. 2a provides a perspective view illustrating the internal configuration of the screw-coil assembly within the thoracostomy tube lumen, showing the relationship between key mechanical transport components. The coils (6) comprise the helical blade structure that extends outwardly from the central rotating core, configured with optimized cupping geometry including concave inner surfaces on the fluid egress side and convex outer surfaces on the fluid inlet side to maximize directional flow characteristics and mechanical transport efficiency. The struts (11) connect the helical blades to the central rotating bar, providing structural integrity while maintaining the open architecture necessary for effective fluid flow between blade segments, with strut orientations configured to optimize fluid movement patterns that complement the primary helical transport action. The central rotating bar (10) serves as the inner core structure of the screw-coil assembly, providing the central axis around which the helical blades are positioned and from which rotational motion is transmitted to create the mechanical conveyance action.

In accordance with an embodiment of the invention, FIG. 2b presents an alternative perspective view of the screw-coil assembly configuration with directional flow indicators, providing enhanced visualization of the three-dimensional relationships between the core mechanical transport elements and operational flow direction. The coils (6) are shown in perspective to demonstrate the helical blade geometry and spatial configuration within the thoracostomy tube environment, illustrating how the cupped blade surfaces create enhanced mechanical force application to fluid and clotted material during rotation. The central rotating bar (10) is depicted to show its relationship to the surrounding helical blade structure and its role as the primary load-bearing and motion-transmission component of the screw-coil assembly. The struts (11) are illustrated in their connecting function between the central rotating bar and the helical blades, demonstrating how the strut configuration maintains the essential transport characteristics while providing the open structure that permits fluid flow between blade segments and enables effective clot transport through positive displacement action. The directional flow indicators demonstrate the drainage inlet (distal end) where fluid and clotted material enter the system and the drainage outlet (proximal end) toward which material is mechanically transported, illustrating the intended operational flow pathway through the drainage system.

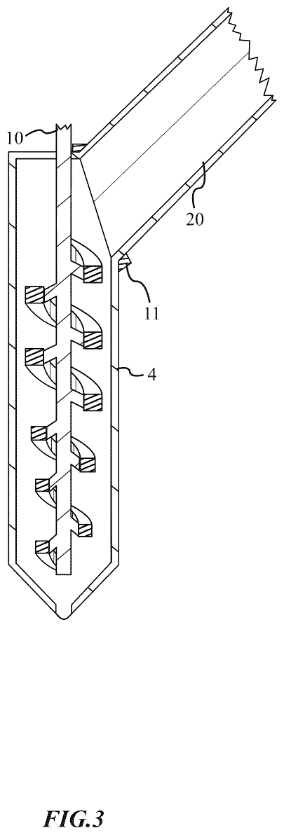

In accordance with an embodiment of the invention, FIG. 3 presents a cross-sectional view that illustrates the dual-lumen configuration of the modified thoracostomy tube, showing the fundamental departure from conventional single-lumen thoracostomy tubes through dedicated pathways for screw-coil operation and sterile drainage. The central rotating bar (10) is positioned within the dedicated screw-coil lumen, demonstrating how the dual-lumen configuration provides contained space for rotational operation without interfering with sterile drainage flow. The side tubes (20) represent the separate drainage pathways within the dual-lumen configuration, specifically dimensioned and positioned to provide adequate cross-sectional area for efficient drainage flow while maintaining separation from the mechanical screw-coil operation. The struts (11) connect the central rotating bar to the helical blade structure, maintaining the open architecture of the screw-coil assembly within the dedicated lumen to enable effective mechanical transport while allowing fluid flow between blade segments. The housing body (4) provides the outer structural envelope of the thoracostomy tube assembly, configured to accommodate both the mechanical clearing system and the essential drainage function, demonstrating the internal lumen separation that enables independent operation of the screw-coil mechanism and the sterile drainage pathway.

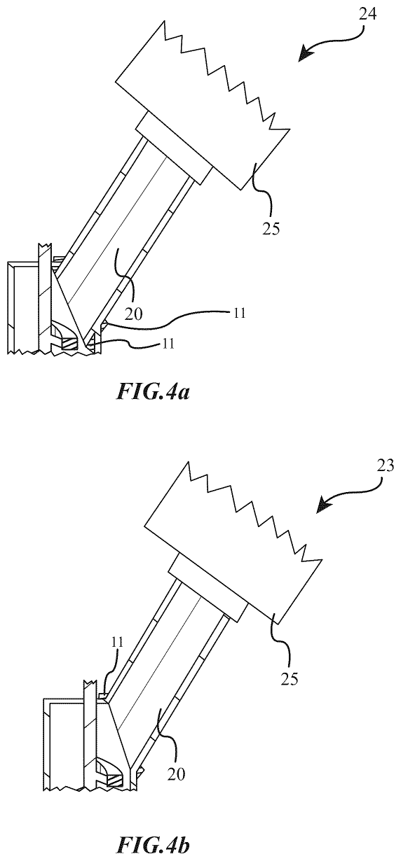

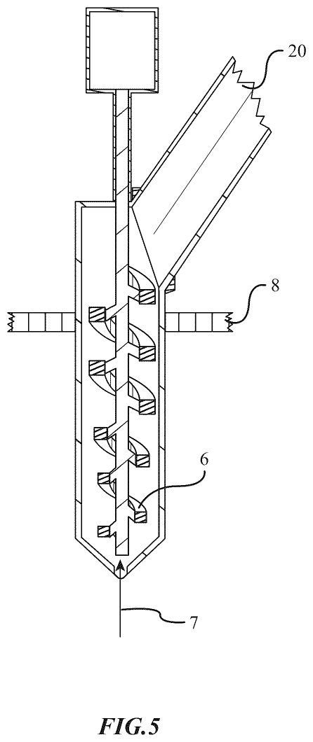

In accordance with an embodiment of the invention, FIG. 4a illustrates a cross-sectional view of the internal tube attachment system and protective configurations, demonstrating the integration of protective features and internal connection mechanisms in accordance with safety embodiments of the invention. The internal tube attachment system (24) represents the internal connection and transition mechanisms that enable the dual-lumen configuration to function effectively within the constrained geometry of the thoracostomy tube, providing smooth transitions between different lumen configurations and maintaining structural integrity throughout the tube length while accommodating the mechanical stresses associated with screw-coil operation. The drainage tube (25) provides the thoracostomy tube structure with integrated protective features designed to prevent tissue infiltration and potential damage during screw-coil operation. The side tubes (20) demonstrate the dual-lumen pathways at the internal connection interface, showing how the dedicated screw-coil lumen and separate drainage outflow maintain their distinct functions through the internal tube architecture. The protective struts (11) are positioned around drainage areas to limit tissue infiltration through the drainage holes, creating barriers that prevent lung tissue from extending into regions where rotating screw-coil contact could cause damage, thereby providing mechanical protection while maintaining drainage flow capability.

In accordance with an embodiment of the invention, FIG. 4b presents a cross-sectional view focusing on the external tube attachment system and the relationship between the dual-lumen components at the external connection interface. The external tube attachment system (23) provides the mechanical interface and sealing mechanisms necessary to maintain sterile barriers and secure connections between the thoracostomy tube assembly and the external motor and drainage collection systems, positioned outside the patient to provide accessible connection points for both the motor-driven screw-coil system and the drainage collection apparatus. The struts (11) extend through the connection region, maintaining the structural connection between the central rotating bar and the helical blade structure while providing the open architecture necessary for effective fluid transport. The drainage tube (25) connects to the external attachment system, demonstrating how the thoracostomy tube interfaces with the external components while maintaining sterile integrity throughout the connection interface. The side tubes (20) are shown in their configuration at the external junction, illustrating how the dual-lumen design provides dedicated pathways that maintain separation between the screw-coil mechanism and the drainage flow through the external connection system.

In accordance with an embodiment of the invention, FIG. 5 shows a cross-sectional view illustrating the integration of the screw-coil mechanism with surrounding tissue and fluid flow pathways, demonstrating how the complete system coordinates mechanical clearing with tissue protection and drainage function. The side tubes (20) demonstrate the dual-lumen configuration within the integrated system, showing how the separate pathways maintain their distinct functions throughout the complete drainage assembly from inlet to collection. The tissue (8) is illustrated in its relationship to the drainage system, showing how lung tissue and other vulnerable structures interact with the thoracostomy tube while being protected by the system's safety features including protective strut configurations and positioning strategies that prevent infiltration into areas where rotating screw-coil contact could cause damage. The housing body (4) provides the thoracostomy tube structure with integrated protective features designed to prevent tissue infiltration and potential damage during screw-coil operation while maintaining adequate drainage flow. The sterile drain chamber (5) provides a contained environment for collecting transported material while maintaining sterile isolation, demonstrating the system configuration during drainage when protective measures and mechanical transport work together. The coils (6) are shown in their operational position within the drainage system, demonstrating how the helical blade geometry creates the mechanical transport action that moves material from the inlet toward the sterile collection chamber. The fluid (7) represents the drainage material flow pathway through the system, illustrating how liquid and clotted material is transported by the screw-coil mechanism and directed into the sterile drain chamber for collection and disposal.

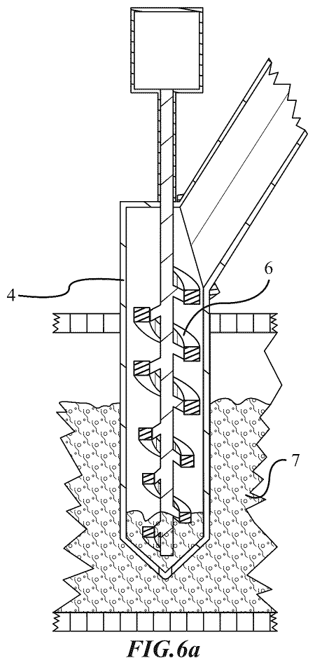

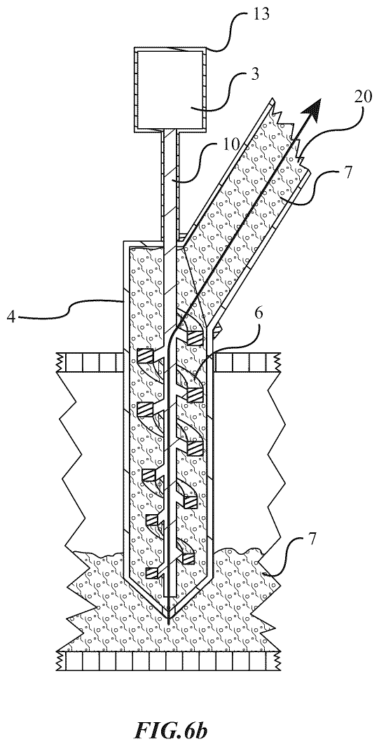

In accordance with an embodiment of the invention, FIG. 6a depicts a cross-sectional view showing the screw-coil assembly and tissue protection features around the drainage area in accordance with embodiments at the commencement of drainage activities. The coils (6) are shown in their initial operational position relative to the fluid and tissue protection features, illustrating how the screw-coil assembly is positioned to provide effective clearing while maintaining safe separation from vulnerable tissue areas during the initial drainage setup when protective measures are most critical. The housing body (4) provides the thoracostomy tube structure with integrated protective features designed to prevent tissue infiltration and potential damage during screw-coil operation, demonstrating the system configuration that accommodates normal drainage flow while providing tissue protection. The fluid (7) represents the initial drainage material that flows through the protective system configuration, demonstrating how the safety features accommodate drainage flow while the screw-coil mechanism prepares to provide mechanical transport capability for clotted material that may develop during the post-operative period.

In accordance with an embodiment of the invention, FIG. 6b illustrates a cross-sectional view of the motor housing and internal components showing the relationship between the screw-coil assembly and dual-lumen thoracostomy tube configuration following commencement of drainage activities. The motor housing (13) encases and protects the motor and associated control electronics while providing a sterile interface between the mechanical actuation system and the thoracostomy tube assembly during active clearing operations. The motor (3) provides continued rotational drive force to maintain tube patency as drainage conditions evolve and clotting patterns develop during the post-operative period, comprising a brushless DC motor with electronic controller for variable speed control and programmable operation cycles. The side tubes (20) maintain their dual-lumen function during active clearing operations, ensuring that mechanical transport and sterile drainage continue to operate independently while providing effective tube patency maintenance. The central rotating bar (10) provides continuous rotational motion transmission to the helical blade structure, enabling sustained mechanical transport throughout the drainage period as clotting conditions vary. The fluid (7) represents the evolving drainage material that requires mechanical transport assistance, illustrating how the screw-coil system adapts to changing fluid characteristics while maintaining effective clearing capability. The housing body (4) maintains structural integrity and protective functions as the system transitions from initial setup to active clearing operations. The coils (6) demonstrate their operational effectiveness in transporting accumulated drainage material, showing how the helical blade geometry continues to provide mechanical conveyance as fluid consistency changes from initial liquid drainage to more viscous or clotted material.

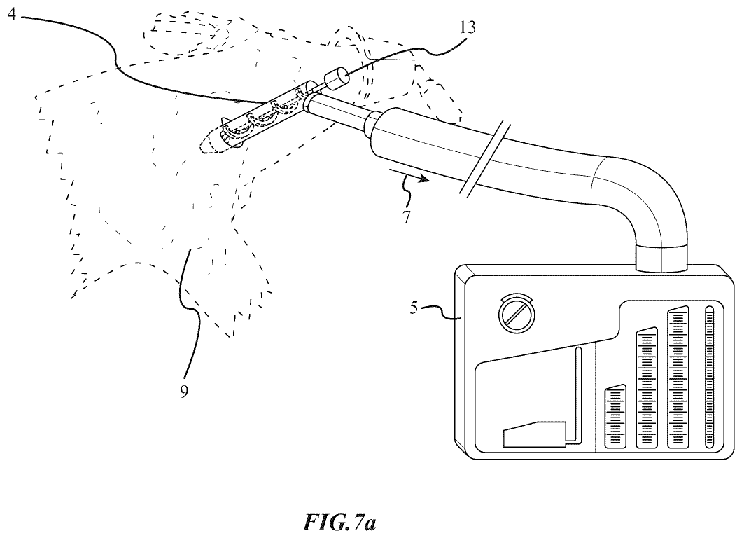

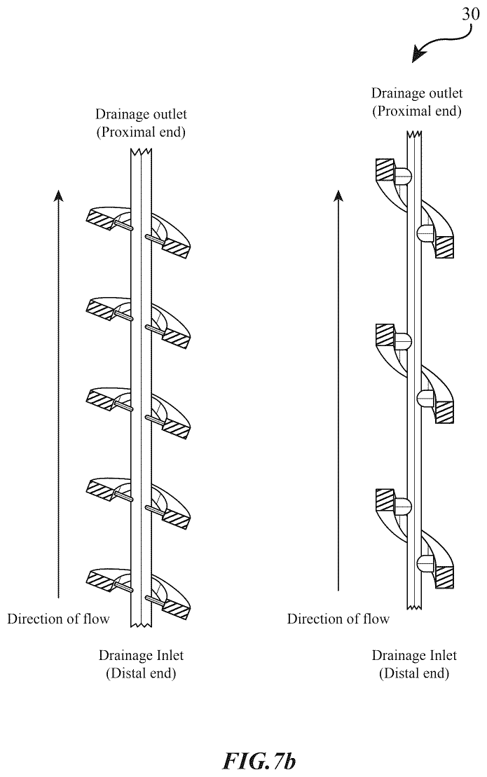

In accordance with an embodiment of the invention, FIG. 7a presents a perspective view showing utilization of the device in accordance with an intended non-human use embodiment of the invention, specifically demonstrating validation testing using animal models to evaluate system performance under physiologically realistic conditions. The housing body (4) maintains the thoracostomy tube structural integrity and protective functions during experimental procedures that evaluate both clearing effectiveness and safety considerations under conditions that closely approximate post-operative clinical scenarios. The motor housing (13) encases the motor and associated control electronics during validation testing, enabling controlled operation of the screw-coil system during experimental evaluation of transport efficiency and system performance. The fluid (7) represents the test medium, typically pig blood or clotted material, used during validation studies to simulate clinical drainage conditions and evaluate the mechanical transport effectiveness of the screw-coil system under controlled experimental parameters. The sterile drain chamber (5) collects transported material during validation testing, enabling quantitative measurement of transport efficiency through volume displacement techniques and providing assessment of system performance under simulated clinical conditions. The animal (pig) (9) provides the physiologically realistic testing environment for in-vivo validation studies that assess system performance under conditions that closely approximate post-operative clinical scenarios, including pressure dynamics, tissue interactions, and drainage conditions that enable comprehensive evaluation of both clearing effectiveness and safety characteristics.

In accordance with an embodiment of the invention, FIG. 7b provides a perspective view with directional flow indicators illustrating alternative configurations and sizing options for the thoracostomy drainage system components in various embodiments. The assorted thread sizes and coil diameter configurations (30) demonstrate the scalability of the inventive drainage system to accommodate the range of thoracostomy tube sizes used in clinical practice, typically ranging from 28 Fr to 32 Fr inner diameter, with screw-coil assembly dimensions, motor specifications, and control system parameters systematically scaled to maintain optimal transport efficiency and safety characteristics across different tube sizes. The directional flow indicators demonstrate the drainage inlet (distal end) where fluid and clotted material enter the system and the drainage outlet (proximal end) toward which material is mechanically transported, illustrating the intended operational flow pathway that remains consistent across different sizing configurations. The alternative configurations show variations in screw pitch (3 mm, 5 mm, and 10 mm), blade diameter configurations representing different percentages of standard thoracostomy tube inner diameter (90%, 75%, and 50%), and operational parameters optimized for different drainage conditions and clotting scenarios, enabling the system to be adapted for specific clinical applications while maintaining the fundamental mechanical transport principles. This configurability ensures that the inventive system can be implemented across the full spectrum of thoracostomy applications while maintaining consistent performance characteristics and providing flexibility to optimize the system for different patient conditions, surgical procedures, and clinical requirements.

In accordance with various embodiments, the invention provides a novel drainage system that utilizes the mechanical principles of an Archimedes screw to transport fluid and clotted material from the drainage inlet toward the outlet. The inventive solution comprises three primary components that work in coordination to maintain thoracostomy tube patency: a screw-coil assembly, a modified thoracostomy tube design, and an automated control system.

The screw-coil assembly, in accordance with an embodiment, represents the core inventive element and comprises an inner core with helical blades connected by struts, configured to rotate within the drainage tube lumen. The screw-coil acts as a mechanical conveyor that can transport clotted blood and other obstructing materials through friction and positive displacement, avoiding the potentially harmful negative pressures generated by conventional suction-based clearing methods.

In accordance with certain embodiments, the modified thoracostomy tube design incorporates features specifically adapted to accommodate the screw-coil assembly while maintaining sterile drainage function. This modified tube design includes dedicated pathways for both the screw-coil mechanism and sterile outflow, representing a departure from conventional single-lumen thoracostomy tubes.

The automated control system, in accordance with various embodiments, provides programmable actuation of the screw-coil assembly through intermittent operation cycles designed to maintain tube patency while minimizing mechanical wear and power consumption. This automation reduces the staff training and maintenance requirements associated with existing active tube clearance systems while providing more economical operation.

In accordance with an embodiment, the invention addresses the fluid dynamics principles that optimize drainage efficiency, incorporating design features such as blade geometry and cupping effects that enhance the mechanical transport of fluid and clotted material from the tube inlet to the outlet.

In accordance with an embodiment, the screw-coil assembly represents the fundamental inventive component that distinguishes the present drainage system from conventional thoracostomy approaches. The screw-coil assembly applies the well-established mechanical principles of an Archimedes screw to the specialized medical application of maintaining thoracostomy tube patency, creating a mechanical conveyance system specifically adapted for transporting fluid and clotted material from the drainage inlet toward the outlet.

In accordance with certain embodiments, the screw-coil assembly comprises an inner core structure with helical blades extending outwardly from the core, with the blades connected to the inner core through a series of structural struts. This configuration allows the screw-coil to maintain the essential transport characteristics of a traditional Archimedes screw while providing enhanced patency compared to conventional solid screw designs. The open structure created by the strut connections permits fluid flow between the blade segments while maintaining the positive displacement action necessary for effective clot transport.

In accordance with an embodiment, the rotational mechanism of the screw-coil assembly operates through continuous or intermittent rotation within the thoracostomy tube lumen. As the screw-coil rotates, the helical blade configuration creates a mechanical conveyor effect that transports clotted blood and other obstructing materials along the length of the tube through a combination of friction and positive displacement forces. This mechanical transport action avoids the potentially harmful negative pressures generated by conventional suction-based clearing methods, which can create dangerous pressure gradients of −87 to −400 cm of water.

In accordance with various embodiments, the screw-coil assembly incorporates specific dimensional parameters that have been optimized through systematic testing to maximize clot evacuation efficiency while maintaining compatibility with standard thoracostomy tube configurations. The screw pitch, defined as the distance between adjacent helical blade turns, represents a critical parameter affecting both the volume of material transported per rotation and the efficiency of fluid flow between blade segments.

In accordance with certain embodiments, screw pitch variations of 3 mm, 5 mm, and 10 mm have been evaluated to determine optimal transport characteristics for different clotting conditions. Testing has demonstrated that tighter pitch configurations (3 mm) provide enhanced transport efficiency for viscous clotted material, while broader pitch configurations (10 mm) optimize flow for liquid drainage while maintaining adequate clot transport capability. The intermediate 5 mm pitch configuration provides balanced performance across varying consistency of drainage material.

In accordance with an embodiment, blade diameter optimization focuses on the relationship between the outer diameter of the helical blades and the inner diameter of the thoracostomy tube. Embodiments have been configured with blade outer diameters representing 90%, 75%, and 50% of the standard thoracostomy tube inner diameter (28 Fr-32 Fr). The 90% diameter configuration maximizes mechanical transport efficiency through enhanced wall contact, while the 75% configuration provides optimal balance between transport efficiency and reduced mechanical stress on the tube walls. The 50% diameter configuration offers reduced friction operation while maintaining adequate clot transport capability.

In accordance with an embodiment, the blade geometry incorporates advanced cupping features specifically designed to optimize fluid dynamics and enhance the mechanical transport efficiency of the screw-coil assembly. These geometric features represent a departure from conventional Archimedes screw designs and address the specific fluid transport challenges encountered in medical drainage applications.

In accordance with certain embodiments, the helical blades incorporate a concave inner surface configuration on the fluid egress side, creating an enhanced cupping effect that increases the mechanical force applied to fluid and clotted material during rotation. This cupping geometry operates on principles similar to those observed in swimming propulsion, where cupped hand positions generate significantly greater propulsive force compared to flat hand orientations. The concave inner surface design enables the rotating blade to more effectively capture and transport drainage material toward the tube outlet.

In accordance with an embodiment, the blade design further incorporates a convex outer surface configuration on the fluid inlet side, creating an optimized fluid capture geometry that enhances the initial engagement between the rotating blade and incoming drainage material. This dual-curvature blade design-concave on the egress side and convex on the inlet side-maximizes the directional flow characteristics that are fundamental to efficient mechanical transport within the constrained geometry of the thoracostomy tube lumen.

In accordance with various embodiments, the strut angle configuration provides additional optimization of fluid movement characteristics. The struts connecting the inner core to the helical blades can be oriented at right angles to the core or angled in directions that optimize fluid flow patterns. Testing has demonstrated that specific strut angle orientations can enhance the overall transport efficiency by creating beneficial flow patterns that complement the primary helical transport action of the blade configuration.

In accordance with an embodiment, the modified thoracostomy tube design incorporates a fundamental departure from conventional single-lumen thoracostomy tubes through the implementation of a dual-lumen configuration specifically adapted to accommodate the screw-coil assembly while maintaining sterile drainage functionality. This dual-lumen design represents a necessary component of the inventive system, as the screw-coil assembly requires dedicated space within the tube structure and must be actuated by external means while preserving the primary drainage function of the thoracostomy tube.

In accordance with certain embodiments, the dual-lumen configuration comprises a dedicated lumen specifically dimensioned and configured for screw-coil insertion and operation. This dedicated lumen provides a contained pathway for the screw-coil assembly to rotate within the thoracostomy tube without interfering with the sterile drainage function. The dedicated lumen is sized to accommodate the outer diameter of the screw-coil assembly while allowing for rotational movement, with appropriate clearance to prevent binding or excessive friction during operation. The dedicated lumen extends substantially along the length of the thoracostomy tube, providing a continuous pathway for screw-coil operation from the external connection point to the drainage inlet region.

In accordance with an embodiment, the dual-lumen configuration further comprises a separate outflow lumen specifically optimized for sterile drainage of fluid and transported material from the pleural space. This separate outflow lumen maintains the essential drainage function of the thoracostomy tube while operating independently of the screw-coil mechanism. The outflow lumen is dimensioned to provide adequate cross-sectional area for efficient drainage flow, accounting for the volume occupied by the dedicated screw-coil lumen within the overall tube structure.

In accordance with various embodiments, the relationship between the dedicated screw-coil lumen and the separate outflow lumen is configured to optimize both mechanical transport efficiency and drainage flow characteristics. The cross-sectional geometry of the dual-lumen configuration can be arranged to provide maximum outflow capacity while maintaining adequate space for screw-coil operation. In certain embodiments, the lumens may be arranged in a side-by-side configuration, while in other embodiments, one lumen may be positioned centrally with the other lumen surrounding it in an annular configuration.

In accordance with an embodiment, Y-connector integration provides the necessary external connection interface for the dual-lumen configuration. The Y-connector serves as the junction point where the dual-lumen thoracostomy tube separates into distinct pathways for screw-coil actuation and sterile drainage outflow. This Y-connector integration enables connection of the screw-coil actuation system to the dedicated lumen while maintaining a separate, sterile pathway for drainage material to flow to the collection system.

In accordance with certain embodiments, the Y-connector can be configured as an external connector positioned outside the patient, providing accessible connection points for both the motor-driven screw-coil system and the drainage collection apparatus. This external Y-connector configuration facilitates easy connection and disconnection of the automated clearing system while maintaining the sterile integrity of the drainage pathway. The external positioning also allows for convenient monitoring and maintenance of the system connections without disturbing the internal thoracostomy tube placement.

In accordance with alternative embodiments, the Y-connector integration may be configured as an internal connector positioned within the thoracostomy tube structure itself, providing a more integrated solution where the dual-lumen separation occurs within the tube body rather than at an external junction. This internal Y-connector embodiment may provide advantages in terms of reduced external connections and potentially improved sterile isolation between the screw-coil mechanism and the drainage pathway.

In accordance with an embodiment, the dual-lumen configuration addresses the fundamental requirement that the inventive drainage system must provide both mechanical clearing capability and sterile drainage function without compromising either aspect. The dedicated screw-coil lumen ensures that the rotating mechanism operates in a controlled environment separate from the drainage flow, while the separate outflow lumen maintains the essential negative pressure and drainage characteristics required for effective thoracostomy function.

This dual-lumen design enables the screw-coil assembly to transport clotted material from the drainage inlet toward the Y-connector junction, where the transported material can be directed into the outflow lumen for removal from the patient, while the screw-coil mechanism itself remains isolated within its dedicated pathway for continued operation.

In accordance with an embodiment, the actuation mechanism provides the rotational drive force necessary to operate the screw-coil assembly within the thoracostomy tube lumen. The actuation mechanism represents a critical component of the inventive system, as it must provide precise control over the rotational speed and timing of the screw-coil operation while maintaining reliable performance in the clinical environment.

In accordance with certain embodiments, the actuation mechanism comprises a brushless DC motor coupled with an electronic controller specifically configured to provide variable speed control and programmable operation cycles. The brushless DC motor configuration offers several advantages over conventional brushed motor designs, including reduced maintenance requirements, enhanced reliability, and improved speed control precision-all critical factors for a medical device intended for automated operation.

In accordance with an embodiment, the motor controller provides electronic regulation of the motor's rotational speed and operational timing. The controller enables precise adjustment of rotational parameters to optimize clot transport efficiency while avoiding excessive mechanical stress on the screw-coil assembly or the thoracostomy tube walls. This electronic control capability allows the system to adapt to varying drainage conditions and clotting patterns encountered in different clinical scenarios.

In accordance with various embodiments, a tachometer system is integrated with the motor assembly to provide real-time verification and measurement of the actual rotational speed (RPM) of the screw-coil assembly. The tachometer serves as a feedback mechanism to ensure that the commanded rotational speed matches the actual operational speed, thereby providing closed-loop control that compensates for variations in mechanical load or motor performance. This feedback control is particularly important when the screw-coil encounters varying resistance due to different consistencies of drainage material or clotted blood.

In accordance with an embodiment, the motor and tachometer system are configured to operate within a rotational speed range of 100 to 20,000 RPM. This broad operational range provides flexibility to optimize the screw-coil performance for different drainage conditions and clotting scenarios. Lower rotational speeds (100-1000 RPM) provide gentle mechanical transport suitable for maintaining tube patency during normal drainage conditions, while higher speeds (10,000-20,000 RPM) can provide enhanced clot morcellization and transport capability when significant obstructions are encountered.

In accordance with certain embodiments, the selection of specific operational speeds within this range is based on systematic testing that has demonstrated optimal clot evacuation efficiency at different RPM settings. Testing has shown that intermediate speeds around 1000 RPM provide effective transport for most clotted material while minimizing mechanical stress, whereas higher speeds approaching 20,000 RPM can morcellize clots at the blade interface, converting larger clot segments into smaller particles that are more easily transported through the drainage system.

In accordance with an embodiment, the programmable automation capability represents a key distinguishing feature that differentiates the inventive system from existing manual active tube clearance devices. The programmable automation addresses the clinical need for maintaining thoracostomy tube patency without requiring constant staff attention or manual intervention, thereby reducing both the training requirements and ongoing maintenance burden associated with tube clearance procedures.

In accordance with various embodiments, the automation system implements intermittent “on/off” cycling of the screw-coil operation to provide efficient clot clearance while reducing mechanical wear on the system components and minimizing power consumption. This intermittent operation recognizes that continuous screw-coil rotation is not necessary to maintain tube patency, and that periodic activation cycles can effectively prevent clot accumulation while extending the operational lifetime of the mechanical components.

In accordance with certain embodiments, the intermittent cycling parameters are programmable to accommodate different drainage conditions and clotting patterns observed in clinical practice. The system can be programmed with specific “on” durations (typically ranging from seconds to minutes) and “off” intervals (ranging from minutes to hours) based on the expected rate of clot formation for particular patient conditions or surgical procedures. This programmability allows the system to be optimized for specific clinical scenarios, such as post-cardiac surgery drainage where clotting rates may differ from thoracic surgery applications.

In accordance with an embodiment, the automated timing is based on clotting patterns that have been characterized through systematic testing using ex-vivo blood clot models and in-vivo validation studies. These studies have provided data on the time intervals required for significant clot formation under different bleeding conditions, enabling the development of timing algorithms that activate the screw-coil system at optimal intervals to prevent tube occlusion before it occurs.

In accordance with various embodiments, the automation system incorporates different operational modes tailored to specific drainage scenarios. For example, a “post-operative bleeding” mode may implement frequent short activation cycles (e.g., 30 seconds every 15 minutes) during the initial hours following surgery when bleeding rates are highest, while a “maintenance” mode may use less frequent cycles (e.g., 60 seconds every 2 hours) during stable drainage conditions.

In accordance with certain embodiments, pressure monitoring integration provides an additional level of automated control and safety monitoring for the screw-coil system. Pressure sensors positioned within the pleural space or at strategic locations within the drainage system can provide real-time feedback on the pressure conditions within the thoracostomy drainage system. This pressure monitoring capability enables the automation system to detect changes in drainage efficiency that may indicate developing occlusions or other system performance issues.

In accordance with an embodiment, the pressure monitoring integration serves both operational and safety functions. From an operational standpoint, pressure changes can trigger automated activation of the screw-coil system when increasing back-pressure indicates developing tube occlusion. From a safety standpoint, pressure monitoring can detect potentially harmful pressure excursions and automatically halt screw-coil operation if pressure levels approach dangerous thresholds that could cause tissue trauma or cardiovascular stress.

In accordance with various embodiments, the automated control system maintains pleural space negative pressure at the prescribed therapeutic levels (typically −20 cmH2O) by coordinating screw-coil operation with the wall suction system. The automation ensures that screw-coil activation does not interfere with the maintenance of appropriate negative pressure gradients required for effective lung expansion and drainage function.

This motor and control system provides the automated, programmable operation necessary to maintain thoracostomy tube patency while addressing the practical clinical requirements for reduced staff burden and enhanced safety compared to existing manual tube clearance approaches.

In accordance with various embodiments, the screw-coil assembly can be manufactured using multiple biocompatible material options, each offering distinct advantages for specific clinical applications and manufacturing requirements. The material selection represents a critical design consideration that affects both the mechanical performance characteristics and the economic viability of the inventive drainage system.

In accordance with certain embodiments, medical-grade stainless steel provides an economical and durable material option for the screw-coil assembly construction. Medical-grade stainless steel offers proven biocompatibility for implantable and indwelling medical devices, while providing the mechanical strength necessary to withstand the rotational stresses encountered during clot transport operations. The economical nature of stainless steel manufacturing makes it particularly suitable for single-use disposable applications, where the screw-coil assembly is disposed of together with the thoracostomy tube after patient use.

In accordance with an embodiment, stainless steel construction provides durability advantages that enable the screw-coil assembly to maintain structural integrity throughout the typical duration of thoracostomy drainage procedures, which may extend from several days to weeks depending on the clinical scenario. The material's resistance to corrosion in the biological environment ensures that the screw-coil assembly maintains consistent performance characteristics throughout the treatment period without degradation that could compromise transport efficiency.

In accordance with alternative embodiments, nitinol (nickel-titanium alloy) offers specialized material properties that provide enhanced flexibility and shape retention characteristics compared to conventional stainless steel construction. Nitinol's superelastic properties enable the screw-coil assembly to accommodate the curved pathways and potential kinking that can occur with thoracostomy tubes during patient movement or positioning changes. This flexibility advantage allows the screw-coil to continue operating effectively even when the thoracostomy tube experiences bending or deformation that might render rigid metal constructions inoperable.

In accordance with an embodiment, the shape retention properties of nitinol enable the screw-coil assembly to return to its original helical configuration after experiencing temporary deformation, thereby maintaining optimal transport efficiency throughout the treatment period. This shape memory capability represents a significant advantage over conventional materials that may experience permanent deformation when subjected to mechanical stress, potentially reducing transport effectiveness or causing complete failure of the clearing mechanism.

In accordance with certain embodiments, while nitinol construction offers superior mechanical properties, it represents a more specialized and costly material option compared to stainless steel alternatives. This cost consideration may make nitinol construction more appropriate for reusable screw-coil assemblies or for applications where the enhanced mechanical properties provide sufficient clinical advantages to justify the increased material costs.

In accordance with various embodiments, biocompatible plastics provide rapid prototyping capability that enables accelerated development and testing of different screw-coil configurations. Biocompatible plastic construction allows for quick production and modification of prototype designs, facilitating iterative optimization of the dimensional parameters such as screw pitch, blade diameter, and strut configurations without the time and expense associated with metal fabrication processes.

In accordance with an embodiment, the rapid prototyping capability of biocompatible plastics enables systematic evaluation of multiple design variations within short timeframes, typically allowing prototype development within one to two weeks compared to the extended lead times required for metal fabrication. This rapid turnaround capability accelerates the design optimization process and enables responsive modifications based on testing results or clinical feedback.

In accordance with certain embodiments, biocompatible plastic construction may come at the cost of reduced durability compared to metal alternatives, potentially limiting the operational lifetime of the screw-coil assembly or requiring more frequent replacement during extended drainage procedures. However, for many thoracostomy applications, the durability of biocompatible plastics may be adequate for the required treatment duration while providing the advantages of reduced manufacturing costs and enhanced design flexibility.

In accordance with various embodiments, the manufacturing approach for the screw-coil assembly incorporates multiple production methods optimized for different phases of development and commercial production. The manufacturing strategy recognizes the distinct requirements for prototype development, design validation, and large-scale production, with specific methods selected to optimize performance, cost, and time-to-market considerations.

In accordance with certain embodiments, 3D printing techniques provide the primary manufacturing method for prototype development and design optimization phases. 3D printing with biocompatible plastics enables rapid production of functional prototypes that can be systematically tested and modified to optimize the screw-coil performance characteristics. This manufacturing approach allows for quick development of prototypes incorporating different dimensional configurations, enabling efficient evaluation of variations in screw pitch, blade diameter, and strut angle parameters.

In accordance with an embodiment, the 3D printing manufacturing process utilizes existing workflows and industry connections to provide cost-effective development of prototypes within accelerated timeframes. The established 3D printing capabilities enable rapid iteration through multiple design variations, with each prototype assigned a unique numeric identifier for reference during testing and evaluation protocols. This systematic approach to prototype development ensures evaluation of design alternatives while maintaining clear documentation of performance characteristics for each configuration.

In accordance with various embodiments, the 3D printing process begins with computer-aided design (CAD) development of the screw-coil geometry, followed by conversion to printable format and fabrication using biocompatible polymer materials. The 3D printed prototypes undergo initial testing to evaluate transport efficiency and mechanical performance before proceeding to more validation testing or advancement to metal fabrication for final production units.

In accordance with certain embodiments, metal fabrication provides the manufacturing method for final production units intended for clinical use. Metal fabrication techniques enable production of screw-coil assemblies using medical-grade stainless steel or nitinol materials with the precision and quality control necessary for medical device applications. The transition from 3D printed prototypes to metal fabrication occurs after design optimization has identified the optimal dimensional parameters and performance characteristics through systematic testing protocols.

In accordance with an embodiment, the metal fabrication process may utilize multiple manufacturing techniques depending on the complexity of the screw-coil geometry and the selected material properties. Conventional machining, electrical discharge machining (EDM), laser cutting, or specialized forming techniques may be employed individually or in combination to achieve the precise dimensional tolerances and surface finishes required for optimal fluid transport performance and biocompatibility.

In accordance with various embodiments, metal fabrication enables production of screw-coil assemblies with enhanced mechanical properties and durability compared to 3D printed alternatives, while maintaining the dimensional precision and performance characteristics validated during prototype testing. The metal fabrication process incorporates appropriate heat treatment, surface finishing, and sterilization preparation to ensure that the final production units meet all regulatory requirements for medical device applications.

In accordance with certain embodiments, quality control considerations represent a critical component of both 3D printing and metal fabrication manufacturing processes. Quality control protocols ensure that each manufactured screw-coil assembly meets the dimensional specifications, mechanical performance requirements, and biocompatibility standards necessary for safe and effective clinical use.

In accordance with an embodiment, quality control procedures include dimensional verification of critical parameters such as screw pitch accuracy, blade diameter consistency, and strut angle precision, all of which directly affect the fluid transport efficiency of the screw-coil assembly. Additional quality control measures encompass surface finish evaluation, mechanical strength testing, and biocompatibility validation to ensure that each production unit maintains consistent performance characteristics and safety profiles.

In accordance with various embodiments, the manufacturing quality control process incorporates statistical process control methods to monitor production consistency and identify any variations that could affect device performance. Documentation systems track each production batch and enable traceability of individual units, supporting regulatory compliance requirements and enabling rapid response to any quality issues that may arise during clinical use.

This manufacturing approach provides the foundation for scalable production of the inventive drainage system while maintaining the quality and performance standards necessary for effective clinical application in maintaining thoracostomy tube patency.

In accordance with various embodiments, the positioning of the screw-coil assembly within the thoracostomy tube represents a critical design parameter that affects both the effectiveness of clot transport and the safety considerations for surrounding lung tissue. Multiple positioning embodiments have been developed to address different clinical scenarios and optimize the balance between mechanical clearing efficiency and tissue protection.

In accordance with an embodiment, the standard positioning configuration locates the screw-coil assembly with its distal end positioned approximately 1 cm from the tube inlet region where drainage holes are located. This standard positioning has been selected based on systematic testing that demonstrates optimal clot transport efficiency while maintaining adequate safety margins for lung tissue protection. The 1 cm positioning places the screw-coil assembly in close proximity to the region where liquid blood is most likely to become clotted, thereby enabling effective mechanical intervention before significant occlusions can develop.

In accordance with certain embodiments, the 1 cm positioning provides an optimal balance between clot clearing effectiveness and operational safety. Testing has demonstrated that positioning the screw-coil closer than 1 cm to the drainage holes may increase the risk of tissue interaction if lung tissue infiltrates the tube through the drainage openings, while positioning the screw-coil further than 1 cm from the drainage holes may reduce the effectiveness of clot prevention in the critical inlet region where clotting is most likely to occur.

In accordance with alternative embodiments, the pre-hole termination embodiment positions the screw-coil assembly to terminate just before the first drainage hole on the tube outlet side. This positioning ensures that the rotating screw-coil never overlaps with any of the drainage holes, thereby eliminating any possibility of tissue damage if lung tissue infiltrates into the tube through the drainage openings. The pre-hole termination embodiment addresses safety concerns by creating a mechanical separation between the rotating screw-coil and any potential tissue infiltration points.

In accordance with certain embodiments, while the pre-hole termination positioning enhances safety by preventing screw-coil overlap with drainage holes, it may allow some clotting to occur in the portion of the tube where the screw-coil is not present. This trade-off between safety and complete clot prevention may be acceptable in clinical scenarios where tissue infiltration risk is elevated or where the consequences of tissue damage would be particularly severe.

In accordance with an embodiment, the tapered coil embodiment incorporates a specialized safety design where the screw-coil assembly is configured with progressively reduced dimensions as it approaches the tube tip region. This tapered configuration, resembling a Christmas tree shape, provides enhanced safety by keeping the rotating screw-coil further from any lung tissue that may infiltrate into the tube while still maintaining adequate clot transport capability in the critical drainage inlet region.

In accordance with various embodiments, the tapered coil design incorporates multiple diameter reductions along the length of the screw-coil as it approaches the drainage holes and tube tip. The progressive tapering provides a graduated safety margin that reduces the risk of tissue damage while maintaining the essential screw-coil transport function. The Christmas tree configuration allows the screw-coil to operate effectively in the proximal regions of the tube while providing increasing levels of tissue protection as the coil approaches the most vulnerable areas near the drainage holes.

In accordance with certain embodiments, the tapered coil embodiment may incorporate different taper angles and reduction ratios depending on the specific clinical application and tube configuration. The taper design can be optimized to provide maximum clot transport efficiency while achieving the required level of tissue protection for the anticipated infiltration risk in specific thoracostomy applications.

In accordance with various embodiments, the thoracostomy tube configuration represents a fundamental component of the inventive system that must accommodate both the screw-coil assembly and the dual-lumen drainage requirements while maintaining compatibility with clinical workflows and safety standards. Multiple tube configuration embodiments provide different approaches to integrating the Y-connector interface and protective features necessary for safe and effective operation.

In accordance with an embodiment, the internal Y-connector embodiment incorporates the dual-lumen separation within the thoracostomy tube structure itself, providing an integrated solution where the division between the screw-coil pathway and the drainage outflow occurs within the tube body rather than at an external junction. This internal Y-connector configuration may provide advantages in terms of reduced external connections and potentially improved sterile isolation between the screw-coil mechanism and the drainage pathway.

In accordance with certain embodiments, the internal Y-connector design enables a more streamlined external interface while maintaining the essential dual-lumen functionality required for screw-coil operation. The internal separation of pathways may reduce the complexity of external connections and potentially minimize the risk of contamination or disconnection during patient care procedures. This configuration may be particularly advantageous in clinical scenarios where minimizing external device complexity is important for ease of use and maintenance.

In accordance with alternative embodiments, the external Y-connector embodiment positions the dual-lumen separation outside the patient, providing accessible connection points for both the motor-driven screw-coil system and the drainage collection apparatus. The external Y-connector configuration facilitates easy connection and disconnection of the automated clearing system while maintaining the sterile integrity of the drainage pathway. This external positioning allows for convenient monitoring and maintenance of system connections without disturbing the internal thoracostomy tube placement.

In accordance with various embodiments, the external Y-connector approach provides operational advantages in terms of system modularity and maintenance accessibility. The external junction enables independent connection of the screw-coil actuation system and the drainage collection system, allowing for system upgrades or maintenance without requiring replacement of the entire thoracostomy tube assembly. This modularity may provide cost advantages and enhanced flexibility in clinical implementation.

In accordance with certain embodiments, the external Y-connector configuration incorporates appropriate sealing and sterilization interfaces to maintain the sterile barrier between the screw-coil mechanism and the drainage pathway. The external junction may include quick-disconnect fittings or other connection mechanisms that facilitate rapid setup and breakdown while ensuring secure and sterile operation throughout the treatment period.

In accordance with various embodiments, protective strut variations provide enhanced safety features to address concerns about lung tissue infiltration into the drainage holes and potential damage from the rotating screw-coil assembly. These protective features represent critical safety enhancements that complement the screw-coil positioning embodiments to provide tissue protection strategies.

In accordance with an embodiment, internal protective strut placement incorporates soft silicon arched or straight struts positioned within the thoracostomy tube to limit tissue infiltration through the drainage holes. These internal struts may be positioned across the drainage hole openings or within the thickness of the tube wall itself to create a barrier that prevents tissue from extending into the tube lumen where it could be damaged by the rotating screw-coil assembly. The internal strut configuration maintains drainage flow while providing mechanical protection against tissue infiltration.

In accordance with certain embodiments, the internal protective struts are manufactured from biocompatible silicon material that provides appropriate flexibility and tissue compatibility while maintaining sufficient structural integrity to prevent tissue infiltration. The struts may be configured as arched structures that span the drainage holes or as straight barriers positioned within the hole openings. The internal strut design must balance the competing requirements of tissue protection, drainage flow optimization, and manufacturing feasibility.

In accordance with alternative embodiments, external protective strut placement incorporates protective structures positioned on the exterior surface of the thoracostomy tube around each drainage hole. These external struts create a mechanical barrier that prevents lung tissue from approaching the drainage hole openings, thereby eliminating the potential for tissue infiltration and subsequent damage from the rotating screw-coil assembly. The external strut configuration may provide enhanced protection while avoiding any internal flow restrictions.

In accordance with various embodiments, the external protective struts may be configured as arch-shaped structures that extend outward from the tube surface around each drainage hole, creating a protective cage that allows fluid flow while preventing tissue contact. The external strut design can accommodate different tissue infiltration risks and may be optimized for specific clinical applications where tissue protection is of particular concern.

In accordance with certain embodiments, combination internal and external protective strut configurations provide maximum tissue protection by incorporating both internal barriers to prevent tissue infiltration and external barriers to prevent tissue approach to the drainage holes. This dual-protection approach may be appropriate for high-risk clinical scenarios where tissue damage consequences would be particularly severe, or where patient anatomy or surgical conditions create elevated infiltration risks.

In accordance with an embodiment, the tube tip design incorporates a cone-shaped configuration that prevents the screw-coil assembly from escaping the distal end of the thoracostomy tube. This cone-shaped tip is tapered to a diameter smaller than the outer diameter of the screw-coil assembly, creating a mechanical retention feature that ensures the screw-coil remains contained within the tube throughout the operational period. The cone-shaped tip diameter may be configured to approximately 1-9 mm depending on the size of the screw-coil assembly, ensuring that the tip opening is narrower than one-third the width of the screw-coil to prevent escape while maintaining adequate drainage flow.

This range of embodiment variations provides flexibility to optimize the inventive drainage system for different clinical scenarios, safety requirements, and operational preferences while maintaining the essential functionality of automated thoracostomy tube patency through mechanical screw-coil transport of clotted material.

In accordance with an embodiment, the clot transport mechanism represents the fundamental operational principle that distinguishes the inventive drainage system from conventional thoracostomy tube clearance approaches. The screw-coil assembly operates as a mechanical conveyor system that provides positive displacement of clotted material and fluid from the drainage inlet toward the outlet without relying on the potentially harmful suction-based methods employed by existing manual clearing techniques.

In accordance with certain embodiments, mechanical conveyance via screw action utilizes the well-established principles of Archimedes screw transport adapted specifically for the medical drainage environment. As the screw-coil assembly rotates within the thoracostomy tube lumen, the helical blade configuration creates a continuous transport mechanism that mechanically moves clotted blood and other obstructing materials along the length of the tube through a combination of friction forces and positive displacement action. This mechanical transport operates independently of the negative pressure gradients that drive the primary drainage function, thereby avoiding interference with the essential pleural space pressure maintenance.

In accordance with an embodiment, the screw conveyor mechanism acts as a rotor that can pass clots between the helical threads through friction, providing effective transport even for viscous or semi-solid drainage material that would resist conventional suction-based clearing methods. The rotating helical blades create a continuously moving surface that engages with clotted material and physically transports it from the inlet region toward the outlet, preventing the accumulation of obstructive deposits that could compromise tube patency.

In accordance with various embodiments, the effectiveness of the mechanical conveyance system is enhanced by the cupping geometry of the helical blades, which optimizes the force of fluid flow through the tube. The concave inner surface of the blades on the fluid egress side creates an enhanced cupping effect that increases the mechanical force applied to both liquid and clotted drainage material during rotation, similar to principles observed in swimming propulsion where cupped hand positions generate significantly greater force than flat orientations.

In accordance with certain embodiments, morcellization of clots at higher speeds provides an additional operational advantage that enhances the overall transport efficiency of the drainage system. When the screw-coil assembly operates at elevated rotational speeds, typically in the range of 10,000 to 20,000 RPM, the mechanical action of the rotating helical blades can break down larger clot segments into smaller particles that are more easily transported through the drainage system.

In accordance with an embodiment, the morcellization process occurs as clots encounter the rotating blade surfaces and are subjected to shear forces that mechanically fragment the clotted material. This fragmentation converts coherent clot masses that might otherwise obstruct the tube into smaller particles that can be more readily transported by both the mechanical screw action and the conventional drainage flow. The morcellization capability provides particular advantage when dealing with well-formed clots that have had sufficient time to develop significant structural integrity.

In accordance with various embodiments, the morcellization effect can be controlled through selection of appropriate rotational speeds and operational timing. Lower speeds (100-1000 RPM) provide gentle mechanical transport that maintains clot integrity while still achieving effective clearance, while higher speeds (10,000-20,000 RPM) actively fragment clots at the blade interface. This controllability allows the system to be optimized for different drainage conditions and clotting scenarios encountered in clinical practice.

In accordance with an embodiment, the avoidance of harmful negative pressure generation represents a critical safety advantage of the mechanical screw transport approach compared to conventional manual clearing methods. Studies have demonstrated that manual stripping techniques can generate mean negative pressures of −87 cm of water when stripping as little as 5 cm of tubing, with full stripping reaching dangerous levels of −400 cm of water. Such excessive negative pressure can cause tissue entrapment, increased bleeding, and left ventricular dysfunction.

In accordance with certain embodiments, the screw-coil transport mechanism operates through positive displacement and friction forces that do not create the harmful pressure gradients associated with suction-based clearing methods. The mechanical transport action moves clotted material through physical contact and conveyance rather than through pressure differentials, thereby avoiding the generation of potentially dangerous negative pressure peaks that could damage surrounding tissue or compromise cardiovascular function.

In accordance with various embodiments, pressure management represents a critical operational consideration that ensures the inventive drainage system maintains the therapeutic negative pressure required for effective thoracostomy function while avoiding potentially harmful pressure excursions during screw-coil operation. The automated control system coordinates screw-coil activation with the maintenance of prescribed pleural space negative pressure levels, typically −20 cmH2O, as required for proper lung expansion and drainage function.

In accordance with an embodiment, maintenance of pleural space negative pressure is achieved through careful coordination between the screw-coil clearing operation and the wall suction system that provides the primary negative pressure gradient. The screw-coil assembly operates within the thoracostomy tube lumen without creating significant resistance to the drainage flow or interfering with the negative pressure transmission from the wall suction source to the pleural space.

In accordance with certain embodiments, the dual-lumen configuration of the modified thoracostomy tube facilitates pressure management by providing separate pathways for screw-coil operation and sterile drainage flow. This separation ensures that mechanical clearing activities do not disrupt the pressure gradients essential for maintaining proper pleural space conditions. The dedicated screw-coil lumen isolates the rotating mechanism from the primary drainage pathway, preventing mechanical interference with pressure maintenance.

In accordance with various embodiments, pressure monitoring integration provides real-time feedback on the pressure conditions within the thoracostomy drainage system, enabling the automated control system to detect and respond to pressure changes that may indicate developing occlusions or other system performance issues. Pressure sensors positioned within the pleural space or at strategic locations within the drainage system can provide continuous monitoring of pressure levels and pressure change rates.

In accordance with an embodiment, the pressure monitoring system serves both operational optimization and safety protection functions. From an operational standpoint, pressure changes can trigger automated activation of the screw-coil system when increasing back-pressure indicates developing tube occlusion, enabling proactive clearing before complete blockage occurs. From a safety standpoint, pressure monitoring can detect potentially harmful pressure excursions and automatically halt screw-coil operation if pressure levels approach dangerous thresholds.

In accordance with certain embodiments, safety considerations in pressure management include establishing upper and lower pressure limits that trigger protective responses from the automated control system. If measured pressures exceed safe operating ranges, either in the positive or negative direction, the system can automatically suspend screw-coil operation and alert clinical staff to potential problems. This automated safety monitoring reduces the risk of pressure-related complications while maintaining effective tube clearing capability.

In accordance with various embodiments, comparison with manual clearing methods demonstrates the pressure management advantages of the automated screw-coil approach. While manual milking and stripping techniques can generate dangerous negative pressures that pose risks to patient safety, the mechanical screw transport operates within controlled pressure parameters that maintain therapeutic effectiveness while avoiding harmful pressure excursions. The positive displacement action of the screw-coil provides effective clot transport without creating the pressure spikes associated with manual clearing techniques.

In accordance with an embodiment, the automated pressure management capability enables the inventive drainage system to maintain consistent pleural space negative pressure throughout the treatment period, even during active clearing operations. This consistency of pressure maintenance represents a significant advantage over manual clearing approaches that can create unpredictable pressure variations and potentially compromise the therapeutic effectiveness of the thoracostomy drainage procedure.

In accordance with certain embodiments, the pressure management system can be programmed to coordinate screw-coil operation with the wall suction cycles, ensuring that clearing activities occur during appropriate phases of the suction cycle to minimize pressure disruption. This coordination optimizes both clearing effectiveness and pressure stability, providing maximum therapeutic benefit while maintaining patient safety throughout the drainage procedure.

In accordance with various embodiments, systematic testing protocols have been developed to evaluate and optimize the performance characteristics of different screw-coil configurations and operational parameters. The ex-vivo testing approach utilizes pig blood clot models that simulate the drainage conditions and clotting patterns encountered in clinical thoracostomy applications, providing quantitative data to guide design optimization and validate the mechanical transport effectiveness of the inventive drainage system.

In accordance with an embodiment, pig blood clot evacuation tests provide the primary methodology for assessing the transport efficiency of different screw-coil designs under controlled conditions that replicate clinical drainage scenarios. These tests utilize fresh pig blood that is allowed to clot within modified thoracostomy tubes containing various screw-coil prototypes, creating realistic obstruction conditions for systematic evaluation of clearing performance. The pig blood model provides appropriate viscosity and clotting characteristics that closely approximate human blood clotting patterns observed in post-operative thoracostomy drainage.

In accordance with certain embodiments, the ex-vivo testing protocol incorporates volume displacement measurements to provide quantitative assessment of screw-coil transport efficiency. Each test involves filling a predetermined volume of pig blood or clotted material at the drainage inlet end of a modified thoracostomy tube, followed by activation of the screw-coil system for designated time periods. The volume of material successfully transported from the inlet to the outlet is measured through water displacement techniques, providing precise quantification of transport effectiveness for each design configuration tested.

In accordance with various embodiments, efficiency comparisons across design variations enable systematic optimization of critical dimensional parameters including screw pitch, blade diameter, and operational speed. Testing protocols compare screw-coil designs with pitch variations of 3 mm, 5 mm, and 10 mm, blade diameter configurations representing 90%, 75%, and 50% of standard thoracostomy tube inner diameter, and operational speeds ranging from 100 to 20,000 RPM. Each permutation undergoes a minimum of 10 trials to establish statistical significance of performance differences and identify optimal design parameters.

In accordance with an embodiment, pleural cavity model testing provides validation of screw-coil performance under physiologically realistic conditions that incorporate the pressure dynamics and tissue interactions present in actual thoracostomy drainage applications. The in-vivo validation utilizes anesthetized pig models with thoracostomy tubes positioned within the pleural space, enabling assessment of both clearing effectiveness and safety considerations under conditions that closely approximate post-operative clinical scenarios.

In accordance with certain embodiments, the in-vivo testing protocol simulates different bleeding conditions through controlled inflow of fresh pig blood into the thoracostomy drainage system. Test conditions include inflow rates of 20 ml per hour, 200 ml per hour, and 450 ml per 24 hours under standard wall suction conditions of −20 cmH2O, representing typical post-operative state, acceptable, and excessive thoracostomy drainage volumes respectively. These controlled bleeding conditions enable evaluation of screw-coil performance across the range of drainage scenarios encountered in clinical practice.

In accordance with various embodiments, pressure change monitoring provides critical safety validation by measuring pleural space pressure variations during screw-coil operation. Pressure sensors positioned alongside the thoracostomy tube inlet monitor pressure changes over time, enabling detection of any potentially harmful pressure excursions that could compromise patient safety. The pressure monitoring protocols compare pressure stability between control conditions (no screw-coil operation) and experimental conditions with screw-coil activation at optimized operational parameters.

In accordance with an embodiment, safety assessment protocols evaluate potential adverse effects on lung tissue and lung function during screw-coil operation. The in-vivo validation includes assessment of tissue infiltration into drainage holes, potential tissue damage from rotating screw-coil contact, and any systemic effects on pulmonary or cardiovascular function. These safety assessments ensure that the inventive drainage system maintains the tissue protection and biocompatibility required for clinical application while providing effective tube clearance capability.

In accordance with various embodiments, the inventive principles of the screw-coil drainage system can be applied to applications beyond thoracostomy tubes to address clotting and obstruction issues in other drainage systems. The mechanical transport principles utilizing Archimedes screw action for positive displacement of fluid and clotted material are adaptable to urinary catheter drainage tubes, smaller surgical drains, and other medical drainage applications where tube patency is compromised by accumulation of viscous or semi-solid material.

In accordance with certain embodiments, applications to other drainage systems may incorporate modified screw-coil designs scaled appropriately for different tube geometries and drainage volumes. Urinary drainage systems, for example, may utilize smaller-diameter screw-coil assemblies with modified pitch and blade configurations optimized for the different fluid characteristics and flow rates associated with urinary drainage compared to thoracostomy applications. The fundamental mechanical transport principles remain applicable across different drainage system categories while allowing for system-specific optimization.

In accordance with an embodiment, scaling for different tube sizes enables the inventive drainage system to accommodate the range of thoracostomy tube sizes used in clinical practice, typically ranging from 28 Fr to 32 Fr inner diameter. The screw-coil assembly dimensions, motor specifications, and control system parameters can be systematically scaled to maintain optimal transport efficiency and safety characteristics across different tube sizes. This scalability ensures that the inventive system can be implemented across the full spectrum of thoracostomy applications while maintaining consistent performance characteristics.

In accordance with various embodiments, integration with existing clinical workflows represents a key design consideration that enables adoption of the inventive drainage system without requiring extensive modifications to established post-operative care procedures. The automated operation and programmable control systems are designed to operate with minimal staff intervention, reducing the training requirements and workflow disruptions associated with implementing new medical technologies. The Y-connector interface enables connection to standard drainage collection systems and wall suction apparatus used in current clinical practice.

In accordance with an embodiment, cost-effectiveness compared to existing active tube clearance (ATC) systems represents a significant advantage of the inventive drainage system. Current ATC devices cost approximately $395 for single-use applications, representing more than 25 times the cost of standard chest tubes. The inventive system utilizes cost-effective materials including medical-grade stainless steel or biocompatible plastics, combined with automated operation that reduces the ongoing costs associated with manual device actuation and maintenance required by existing ATC systems.

In accordance with certain embodiments, reduced staff training and maintenance requirements provide practical advantages that facilitate clinical adoption and reduce operational costs. The automated, programmable operation eliminates the need for staff to manually actuate clearing devices or perform routine maintenance procedures associated with existing ATC systems. The self-contained design and intermittent operational cycles minimize mechanical wear and extend operational lifetime, reducing the maintenance burden on clinical staff and improving the reliability of tube clearance function throughout the treatment period.

In accordance with various embodiments, improved patient outcomes through maintained tube patency address the fundamental clinical problem that the inventive drainage system is designed to solve. By providing reliable, automated prevention of tube occlusion, the system enables continuous evacuation of blood and fluid from the pleural space, maintaining the negative pressure gradients essential for proper lung expansion and preventing the accumulation of retained blood that can induce inflammation and fibrosis. The maintained tube patency reduces the risk of post-operative complications including pneumothorax, decreased lung capacity, and cardiac tamponade that result from inadequate drainage function.

In accordance with the various embodiments described herein, the present invention provides a solution to the persistent clinical challenge of thoracostomy drainage tube obstruction through an integrated system comprising a screw-coil assembly, a modified thoracostomy tube design, and an automated control system. The inventive approach utilizes the mechanical principles of Archimedes screw transport to provide positive displacement of clotted blood and other obstructing materials from the drainage inlet toward the outlet, avoiding the potentially harmful negative pressures generated by conventional manual clearing methods while maintaining the essential negative pressure required for therapeutic drainage function. The screw-coil assembly incorporates advanced blade geometry with cupping effects that optimize fluid transport efficiency, while the dual-lumen thoracostomy tube design provides dedicated pathways for both screw-coil operation and sterile drainage outflow. Multiple safety features including protective struts, tapered coil configurations, and appropriate termination positioning ensure tissue protection while maintaining effective clearing capability. The automated control system enables programmable intermittent operation based on clotting patterns, reducing staff training and maintenance requirements while providing cost-effective tube clearance that addresses the practical limitations of existing active tube clearance technologies. Through systematic testing protocols and validation studies, the invention has been demonstrated to provide reliable maintenance of tube patency while offering significant economic and operational advantages over current solutions, ultimately improving patient outcomes through enhanced drainage function and reduced post-operative complications.

In accordance with an embodiment, the novel surgical drainage tube design represents a fundamental inventive element that distinguishes the present system from prior art approaches. Unlike conventional drainage tubes that comprise simple cylindrical structures with conical distal ends and apertures smaller than the tube diameter, the inventive tube design incorporates a specialized proximal end configuration specifically engineered to accommodate the screw-coil assembly while maintaining preserved dedicated outflow for sterile drainage collection. This novel proximal end design creates a departure from standard drainage tube geometry and enables the integration of mechanical clearing capability without compromising drainage function.

In accordance with certain embodiments, the proximal end of the novel surgical drainage tube comprises a dedicated entry point specifically dimensioned and configured for screw-coil insertion. This dedicated entry point provides coaxial alignment with the tube lumen to enable substantially straight insertion of the screw-coil assembly into the drainage tube, minimizing insertion complexity and enabling reliable mechanical engagement between the screw-coil mechanism and the tube interior. The dedicated entry point geometry is configured to provide appropriate clearance for the screw-coil outer diameter while maintaining sufficient structural integrity to support rotational operation without tube deformation or failure.

In accordance with an embodiment, the proximal end configuration further comprises a separately preserved dedicated outflow lumen that maintains sterile drainage collection capability independent of the screw-coil insertion pathway. This separately preserved outflow represents a critical design feature that enables drainage material transported by the screw-coil mechanism or flowing naturally through the tube to be efficiently diverted to external collection systems while the mechanical clearing system continues to operate. The separately preserved nature of this outflow pathway ensures that drainage function is not compromised by screw-coil operation and that sterile isolation can be maintained between the mechanical components and the collected drainage material.

In accordance with various embodiments, the relationship between the dedicated screw-coil entry point and the separately preserved outflow lumen is configured through or similar conceptually to a Y-connector integration at the proximal end of the drainage tube. This Y-connector configuration provides the interface geometry necessary to separate the incoming drainage flow from the screw-coil insertion pathway, directing drainage material to the collection system while maintaining dedicated access for the mechanical clearing mechanism. The Y-connector design represents a critical element of the novel tube configuration and enables the coordinated operation of mechanical clearing and sterile drainage collection.

In accordance with an embodiment, the Y-connector configuration at the proximal end of the surgical drainage tube provides optimized fluid diversion mechanics that naturally direct drainage material to the collection pathway while maintaining dedicated screw-coil access. The Y-connector design resembles urinary catheter Y-end configurations, where fluid flowing through a primary conduit encounters a junction that diverts flow to a separate outflow port through geometric features that favor fluid separation over continued flow in the original direction.

In accordance with certain embodiments, the Y-connector incorporates a coaxial outflow port aligned substantially with the drainage tube axis, configured with a wider cross-sectional area and soft angular transition that naturally captures drainage material flowing proximally through the tube. As fluid and transported clotted material move from the distal end toward the proximal end under the influence of the screw-coil transport mechanism or natural drainage pressure gradients, the fluid encounters the Y-junction where the geometric configuration of the wider soft-angled outflow port creates a preferential flow path that diverts drainage material away from the screw-coil insertion pathway.

In accordance with an embodiment, the outflow port is configured more coaxially with the drainage tube than the screw-coil insertion port, creating a natural flow preference where drainage material continues substantially along its original flow trajectory into the collection pathway rather than requiring sharp directional changes. This coaxial alignment minimizes flow resistance and enables efficient drainage diversion without creating back-pressure that could compromise drainage function or create turbulent flow patterns that might reduce transport efficiency.

In accordance with various embodiments, the screw-coil insertion port of the Y-connector provides dedicated access oriented at an angle relative to the primary drainage tube axis, enabling screw-coil insertion while maintaining separation from the drainage flow pathway. The angular orientation of the insertion port may range from approximately 15 degrees to 90 degrees relative to the tube axis, selected to optimize ease of screw-coil insertion and maintenance accessibility while ensuring that the insertion pathway does not interfere with efficient drainage diversion to the outflow port.

In accordance with certain embodiments, the Y-connector internal geometry incorporates smooth transitions and optimized junction angles that minimize turbulent flow and pressure drops as drainage material transitions from the primary tube lumen to the outflow port. The junction geometry may incorporate gradual radius transitions, streamlined divider surfaces, and appropriate cross-sectional area ratios that maintain drainage efficiency comparable to single-lumen conventional tubes while providing the additional functionality of dedicated screw-coil access.

In accordance with an embodiment, alternative Y-connector configurations may position the outflow port at various orientations relative to the drainage tube axis while maintaining the fundamental principle of providing naturally preferential flow diversion to the collection pathway. Embodiments may include lateral outflow configurations, angled outflow configurations ranging from 15 to 45 degrees from the tube axis, or other geometric arrangements that achieve efficient fluid diversion based on the specific clinical application requirements and connection interface preferences.

In accordance with an embodiment, the handedness of the helical coil represents a critical design parameter that determines the direction of fluid transport relative to the rotational direction of the screw-coil assembly. Helical coil handedness refers to the orientation of the helical thread as it wraps around the central rotating bar, defined by whether the helix follows a right-hand thread pattern or a left-hand thread pattern. This handedness, in combination with the rotational direction, determines whether the mechanical transport action moves material from the distal end toward the proximal end (desired) or from the proximal end toward the distal end (undesired).

In accordance with certain embodiments, a right-hand helical coil configuration comprises helical blades that wrap around the central rotating bar in a pattern matching the thread orientation of conventional right-hand screw threads. When viewing the screw-coil assembly from the proximal end (outside the patient) toward the distal end (inside the patient), a right-hand helix rises in a clockwise direction as it extends away from the viewer. For a right-hand helical coil to create distal-to-proximal transport (from deep in the patient toward the outside), the screw-coil must rotate in a counter-clockwise direction when viewed from the proximal end.

In accordance with an embodiment, a left-hand helical coil configuration comprises helical blades that wrap around the central rotating bar in a pattern matching the thread orientation of left-hand screw threads, opposite to conventional right-hand threads. When viewing the screw-coil assembly from the proximal end toward the distal end, a left-hand helix rises in a counter-clockwise direction as it extends away from the viewer. For a left-hand helical coil to create distal-to-proximal transport, the screw-coil must rotate in a clockwise direction when viewed from the proximal end.

In accordance with various embodiments, the selection of coil handedness is coordinated with the motor rotational direction and control system programming to ensure consistent distal-to-proximal transport across all operational conditions. The automated control system is programmed to rotate the screw-coil assembly in the appropriate direction based on the handedness of the installed coil, ensuring that fluid and clotted material are always transported from the drainage inlet (distal end) toward the drainage outlet (proximal end) regardless of which handedness configuration is employed.

In accordance with certain embodiments, the preferred handedness configuration comprises a left-hand helical coil operated with clockwise rotation when viewed from the proximal end, providing intuitive operational characteristics where clockwise rotation produces outward transport of material from the patient. This configuration aligns with conventional understanding of clockwise rotation as producing forward or outward motion, potentially simplifying operator understanding and system setup procedures.

In accordance with alternative embodiments, a right-hand helical coil operated with counter-clockwise rotation when viewed from the proximal end provides equivalent distal-to-proximal transport functionality. The selection between right-hand and left-hand configurations may be based on manufacturing preferences, motor characteristics that favor particular rotational directions, or clinical workflow considerations that make one configuration more intuitive or convenient than the other.

In accordance with an embodiment, the importance of correct handedness configuration cannot be overstated, as incorrect matching of coil handedness to rotational direction would result in proximal-to-distal transport that pushes material deeper into the patient rather than extracting it toward the collection system. To prevent such incorrect operation, the system may incorporate verification mechanisms including visual indicators on the screw-coil assembly marking the handedness, control system programming that requires handedness confirmation before operation, or mechanical keying features that prevent installation of incorrect handedness coils with incompatible motor rotation configurations.

In accordance with an embodiment, the strut angle relative to the central rotating bar represents a critical design parameter that significantly affects the fluid flow dynamics and transport efficiency of the screw-coil assembly. Unlike conventional Archimedes screws where struts may be oriented perpendicular to the central axis (90 degrees), the optimized strut angles of the present invention are configured at acute angles less than 90 degrees relative to the central rotating bar, specifically angled toward the distal end of the drainage tube to create flow patterns that favor distal-to-proximal fluid movement.

In accordance with certain embodiments, strut angles in the range of 30 to 80 degrees relative to the central bar axis provide enhanced fluid capture and directional guidance during rotational operation. When the struts angle backward toward the distal end (the direction from which fluid originates), the rotating strut surfaces encounter incoming fluid at angles that promote capture and forward transport. This angled orientation creates scooping action that complements the primary helical blade transport, enhancing overall system efficiency compared to perpendicular strut orientations that may create flow resistance or turbulence.

In accordance with an embodiment, strut angles of approximately 45 degrees relative to the central bar axis provide optimal balance between structural integrity, manufacturing feasibility, and fluid transport enhancement. At this intermediate angle, the struts provide sufficient backward lean to create beneficial flow capture effects while maintaining adequate load-bearing capacity to withstand the mechanical stresses of rotational operation and clot transport resistance. Testing has demonstrated that 45-degree strut angles provide measurable improvements in transport efficiency compared to perpendicular strut configurations.

In accordance with various embodiments, steeper strut angles (closer to the central bar axis, such as 30-degree angles) provide enhanced fluid capture effects and more pronounced directional flow guidance, potentially beneficial for high-viscosity drainage material or heavily clotted conditions where maximum transport force is desired. However, steeper angles may reduce the structural load-bearing capacity of the struts and may create manufacturing challenges depending on the fabrication method employed.

In accordance with certain embodiments, more gradual strut angles (further from the central bar axis, such as 60 to 80 degrees) provide enhanced structural strength and simplified manufacturing while still offering fluid flow advantages compared to perpendicular orientations. These more gradual angles may be preferred in applications where mechanical durability is prioritized or where manufacturing methods favor less extreme angular features.

In accordance with an embodiment, the strut angle may vary along the length of the screw-coil assembly to optimize performance in different regions of the drainage tube. Struts near the distal end where fluid first enters the system may employ steeper angles (30-40 degrees) to maximize initial fluid capture, while struts in the mid-section may employ intermediate angles (45-55 degrees) for balanced transport, and struts near the proximal end may employ more gradual angles (60-70 degrees) where structural strength to resist discharge forces is prioritized.

In accordance with various embodiments, the strut angle optimization coordinates with the coil handedness to ensure that the angular orientation consistently favors distal-to-proximal transport. The backward lean of the struts toward the distal end, combined with appropriate helical handedness and rotational direction, creates synergistic transport enhancement where both the primary helical action and the secondary strut flow effects work in concert to maximize transport efficiency.

In accordance with an embodiment, the configuration of drainage pathways through the surgical drainage tube represents a design consideration that may be optimized based on the specific geometry and operational characteristics of different clinical applications. The invention encompasses both single-aperture embodiments where drainage and screw-coil insertion share a common proximal aperture with Y-connector diversion, and dual-lumen embodiments where separate pathways are maintained throughout the tube length.

In accordance with certain embodiments where the screw-coil assembly can be inserted substantially straight into the drainage tube without requiring curved or complex insertion pathways, the single-aperture configuration with proximal Y-connector diversion provides optimal simplicity and drainage efficiency. In this configuration, the drainage tube comprises a single primary lumen throughout its length from the distal drainage holes to the proximal Y-connector junction. The screw-coil assembly inserts through the dedicated entry port of the Y-connector and extends substantially straight through the single lumen to its operational position near the distal drainage holes.

In accordance with an embodiment, the single-aperture configuration provides significant advantages in terms of manufacturing simplicity, maximized drainage flow cross-sectional area, and elimination of internal lumen separators that could create flow resistance or complicate tube fabrication. Because the screw-coil and the drainage flow share the same lumen with separation occurring only at the proximal Y-connector, there is no benefit to separating the two lumens for fluid flow dynamics within the tube body. The drainage material flows proximally through the same lumen occupied by the screw-coil, with the Y-connector geometry providing natural fluid diversion to the outflow port as described previously.

In accordance with various embodiments, the single-aperture configuration requires that the screw-coil design incorporate sufficient open structure to allow drainage flow between and around the helical blades. The strut-connected blade design of the present invention provides this open architecture, enabling fluid to flow through the spaces between blade segments while the mechanical transport action moves clotted material proximally. The screw-coil thus operates within the flowing drainage stream without creating significant flow obstruction.

In accordance with alternative embodiments, a dual-lumen configuration throughout the tube length may provide advantages in specific clinical scenarios where enhanced sterile isolation between the mechanical components and the drainage flow is desired, or where the screw-coil assembly requires curved or complex insertion pathways that would benefit from dedicated lumen separation. In these dual-lumen embodiments, the drainage tube incorporates internal separation structures that create distinct pathways for the screw-coil mechanism and the drainage flow throughout the tube length from proximal end to distal end.

In accordance with certain embodiments, the dual-lumen configuration comprises a central lumen dedicated to screw-coil operation, surrounded by an annular lumen dedicated to drainage flow. This concentric configuration provides symmetric drainage flow around the central screw-coil pathway and may offer manufacturing advantages for certain tube fabrication methods. Alternative dual-lumen configurations may employ side-by-side lumen arrangements where the screw-coil lumen and drainage lumen are positioned adjacent to each other within the tube cross-section.

In accordance with an embodiment, the selection between single-aperture and dual-lumen configurations is based on the specific requirements of the surgical drainage application, the tube diameter and length considerations, the screw-coil insertion geometry, and the desired level of sterile isolation between mechanical components and drainage flow. Both configurations are encompassed within the scope of the present invention and provide effective solutions to the fundamental problem of maintaining surgical drainage tube patency through automated mechanical clearing.

In accordance with various embodiments, the inventive principles of the gravity-independent surgical drainage system extend beyond thoracostomy applications to address clotting and obstruction challenges across multiple surgical drainage contexts. The fundamental mechanical transport principles utilizing directional Archimedes screw action for positive displacement of fluid and clotted material are applicable to any surgical drainage scenario where tube patency is compromised by accumulation of viscous or coagulated material, and where gravity-independent transport capability provides clinical advantages.

In accordance with certain embodiments, abdominal surgical drainage applications represent a significant opportunity for the inventive drainage system. Following abdominal surgeries including hepatic resection, pancreatic surgery, colorectal procedures, and general abdominal trauma repairs, drainage tubes are routinely placed to evacuate blood, serous fluid, and other drainage material from the peritoneal cavity. These abdominal drains commonly experience clotting and obstruction that compromises drainage function, leading to fluid accumulation, abscess formation, and other post-operative complications. The gravity-independent transport capability of the inventive screw-coil system enables effective drainage regardless of patient positioning and provides maintained tube patency throughout the post-operative recovery period.

In accordance with an embodiment, pericardial drainage applications following cardiac surgery or pericardial procedures represent another important clinical context where the inventive drainage system provides significant advantages. Pericardial effusions containing blood or serous fluid must be effectively evacuated to prevent cardiac tamponade and ensure proper cardiac function. Conventional pericardial drainage tubes are susceptible to clotting and obstruction that can create life-threatening complications. The automated mechanical clearing capability of the inventive system maintains pericardial drainage tube patency and enables reliable evacuation of pericardial fluid throughout the treatment period.

In accordance with various embodiments, pleural effusion drainage beyond post-operative thoracostomy represents an additional application where the inventive system provides clinical benefits. Pleural effusions from malignant disease, infection, or other non-surgical causes require drainage tubes that must remain patent for extended periods. The chronic nature of many pleural effusion conditions creates particular challenges for tube patency maintenance, as drainage tubes may remain in place for weeks or months rather than the typical few days of post-operative drainage. The automated operation and programmable control of the inventive system make it particularly suitable for these extended-duration drainage scenarios.

In accordance with certain embodiments, orthopedic surgical drainage following joint replacement procedures, spinal surgery, or orthopedic trauma repairs represents another application context where drainage tube clotting compromises post-operative care. Blood accumulation in surgical sites can create complications including infection risk, delayed healing, and impaired joint function. The scalable design of the inventive drainage system enables adaptation to the smaller tube sizes commonly used in orthopedic drainage while maintaining effective mechanical clearing capability.

In accordance with an embodiment, neurosurgical drainage applications including ventricular drainage and subdural drainage represent specialized contexts where tube patency is critical and where clotting can create severe complications. While neurosurgical drainage tubes are typically smaller diameter than thoracostomy or abdominal drains, the fundamental principles of the inventive system remain applicable with appropriate scaling of screw-coil dimensions and operational parameters to suit the specific requirements of neurosurgical drainage applications.

In accordance with various embodiments, urological drainage applications represent an additional opportunity for the inventive drainage system, particularly in post-surgical settings where bloody urine or clot formation can obstruct urinary drainage tubes. The mechanical transport principles of the screw-coil assembly can be adapted to urological drainage tube geometries and flow characteristics, providing maintained tube patency in applications where conventional irrigation-based clearing methods may be inadequate or contraindicated.

In accordance with certain embodiments, the scalability and adaptability of the inventive drainage system across these multiple clinical contexts is achieved through systematic adjustment of key design parameters including tube diameter, screw-coil blade diameter and pitch, motor specifications, and control system operational parameters. The fundamental mechanical transport principles remain consistent across applications while the specific implementation is optimized for each surgical drainage context. This broad applicability significantly expands the clinical utility and commercial potential of the inventive drainage system beyond the initially developed thoracostomy application.

In accordance with the present invention, consistent terminology is employed throughout this specification to clearly identify anatomical relationships and directional flow characteristics of the surgical drainage system. The term “distal end” refers to the portion of the drainage tube that is positioned deepest within the patient's body cavity where drainage fluid enters the system through drainage apertures. The term “proximal end” refers to the portion of the drainage tube that remains outside the patient's body where connections are made to the screw-coil actuation system and to the drainage collection apparatus. This is shown in accordance with an embodiment in FIG. 7b.

In accordance with this directional convention, fluid flow through the drainage system proceeds from the distal end toward the proximal end, corresponding to flow from the internal body cavity outward to the external collection system. This distal-to-proximal flow direction represents the natural and desired direction for surgical drainage applications and establishes the reference frame for describing the operational characteristics of the screw-coil transport mechanism.

In accordance with an embodiment, the screw-coil assembly is specifically configured to create mechanical transport in the distal-to-proximal direction through appropriate matching of helical coil handedness with rotational direction as described in detail in previous sections. All references to transport direction, fluid movement, and directional flow characteristics in this specification employ the distal-to-proximal convention unless explicitly stated otherwise.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

I claim:

1. A gravity-independent surgical drainage system for maintaining tube patency, comprising:
a surgical drainage tube comprising:
a tube body having a distal end configured for insertion into a patient body cavity with drainage apertures for fluid inlet and a proximal end configured to remain outside the patient;
a proximal end configuration comprising a dedicated entry point for screw-coil insertion and a separately preserved dedicated outflow lumen for sterile drainage collection;
a directional screw-coil assembly configured for insertion through said dedicated entry point and extension into said tube body toward said distal end, said screw-coil assembly comprising:
a central rotating bar extending along a longitudinal axis;
helical blades spiraling around said central rotating bar with defined handedness selected from right-hand helical configuration and left-hand helical configuration;
structural struts connecting said helical blades to said central rotating bar at strut angles less than 90 degrees relative to said longitudinal axis, said struts angled toward said distal end to favor fluid flow from said distal end toward said proximal end;
wherein said helical blades comprise a concave inner surface on a fluid egress side and a convex outer surface on a fluid inlet side;
a motor and control system configured to:
rotate said screw-coil assembly in a rotational direction matched to said helical blade handedness to create mechanical transport of fluid and clotted material from said distal end toward said proximal end; and
provide programmable intermittent operation cycles to maintain tube patency.

2. The gravity-independent surgical drainage system of claim 1, wherein said proximal end configuration comprises a Y-connector providing fluid diversion, said Y-connector comprising:
a screw-coil insertion port providing said dedicated entry point; and
an outflow port configured more coaxially with said tube body than said screw-coil insertion port, said outflow port having a wider cross-sectional area and soft angular transition that naturally captures drainage material flowing proximally through said tube body and diverts said material to a drainage collection pathway.

3. The gravity-independent surgical drainage system of claim 2, wherein said tube body comprises a single primary lumen extending from said distal end to said Y-connector, wherein drainage material and said screw-coil assembly share said single primary lumen with separation between screw-coil pathway and drainage collection pathway occurring at said Y-connector.

4. The gravity-independent surgical drainage system of claim 1, wherein said screw-coil assembly comprises a left-hand helical configuration, and wherein said motor and control system is configured to rotate said screw-coil assembly in a clockwise direction when viewed from said proximal end toward said distal end to create said mechanical transport from said distal end toward said proximal end.

5. The gravity-independent surgical drainage system of claim 1, wherein said structural struts are oriented at strut angles in a range of 30 to 80 degrees relative to said longitudinal axis of said central rotating bar.

6. The gravity-independent surgical drainage system of claim 5, wherein said strut angles are approximately 45 degrees relative to said longitudinal axis to provide optimized balance between fluid capture enhancement and structural integrity.

7. The gravity-independent surgical drainage system of claim 1, wherein said surgical drainage tube is configured for applications selected from thoracostomy drainage, abdominal cavity drainage, pericardial drainage, pleural effusion drainage, orthopedic drainage, or urological drainage.

8. The gravity-independent surgical drainage system of claim 1, wherein said motor and control system comprises:
a brushless DC motor with electronic controller configured to provide variable speed control within a range of 100 to 20,000 RPM; and
control logic that matches rotational direction to said helical blade handedness to ensure consistent distal-to-proximal transport.

9. The gravity-independent surgical drainage system of claim 8, wherein said control system is configured to operate said screw-coil assembly at lower rotational speeds of 100 to 1000 RPM for gentle mechanical transport and at higher rotational speeds of 10,000 to 20,000 RPM for clot morcellization.

10. The gravity-independent surgical drainage system of claim 1, wherein said helical blades have an outer diameter representing 75% to 90% of an inner diameter of said tube body to maximize mechanical transport efficiency while minimizing friction resistance.

11. The gravity-independent surgical drainage system of claim 1, further comprising protective features positioned around said drainage apertures at said distal end, said protective features comprising struts or barriers configured to prevent tissue infiltration through said drainage apertures into proximity with said rotating screw-coil assembly.

12. A surgical drainage tube for use with automated mechanical clearing systems, comprising:
a tube body extending from a distal end configured for insertion into a patient body cavity to a proximal end configured to remain outside the patient;
drainage apertures at said distal end for fluid inlet from said body cavity;
a proximal end configuration comprising:
a dedicated entry point specifically dimensioned for insertion of a screw-coil clearing mechanism, said entry point providing substantially straight access into said tube body toward said distal end; and
a separately preserved dedicated outflow lumen maintaining sterile drainage collection capability independent of said entry point;
wherein said proximal end configuration comprises a Y-connector that naturally diverts drainage flow to said outflow lumen while maintaining dedicated screw-coil access.

13. The surgical drainage tube of claim 12, wherein said Y-connector comprises:
a screw-coil insertion port providing said dedicated entry point;
an outflow port configured more coaxially with said tube body axis than said screw-coil insertion port; and
internal geometry creating preferential flow diversion to said outflow port for drainage material flowing proximally through said tube body.

14. The surgical drainage tube of claim 12, wherein said tube body comprises a single primary lumen extending from said distal end to said Y-connector, enabling shared usage by a screw-coil assembly and drainage flow with separation occurring at said proximal Y-connector.

15. The surgical drainage tube of claim 12, further comprising an alternative dual-lumen configuration throughout said tube body length, comprising a dedicated screw-coil lumen and a separate drainage lumen that are fluidly isolated throughout said tube body length.

16. The surgical drainage tube of claim 12, further comprising protective structures positioned around said drainage apertures to prevent tissue infiltration into said tube body and a cone-shaped tip at said distal end tapered to prevent escape of a screw-coil clearing mechanism.

17. A directional screw-coil assembly for surgical drainage tube clearing, comprising:
a central rotating bar extending along a longitudinal axis configured for rotational actuation;
helical blades spiraling around said central rotating bar with defined handedness selected from right-hand or left-hand helical configuration, said handedness coordinated with intended rotational direction to create fluid transport from a distal drainage inlet toward a proximal drainage outlet;
structural struts connecting said helical blades to said central rotating bar at acute strut angles less than 90 degrees relative to said longitudinal axis, said struts angled backward toward an intended distal position to favor distal-to-proximal fluid flow;
wherein said helical blades comprise a concave inner surface on a fluid egress side creating enhanced cupping effect and a convex outer surface on a fluid inlet side creating optimized fluid capture geometry.

18. The directional screw-coil assembly of claim 17, wherein said strut angles are in a range of 30 to 80 degrees relative to said longitudinal axis.

19. The directional screw-coil assembly of claim 17, wherein said strut angles are approximately 45 degrees to provide optimized balance between fluid capture enhancement and structural integrity.

20. The directional screw-coil assembly of claim 17, wherein said helical blades are configured with screw pitch selected from 3 mm, 5 mm, or 10 mm between adjacent blade turns to optimize transport efficiency for different drainage material viscosities.

21. The directional screw-coil assembly of claim 17, comprising a left-hand helical configuration intended for clockwise rotation to create distal-to-proximal transport, or comprising a right-hand helical configuration intended for counter-clockwise rotation to create distal-to-proximal transport.

22. The directional screw-coil assembly of claim 17, wherein said strut angles vary along a length of said screw-coil assembly, comprising steeper strut angles of 30 to 40 degrees near said distal position for maximum fluid capture, intermediate strut angles of 45 to 55 degrees in a mid-section for balanced transport, and more gradual strut angles of 60 to 70 degrees near a proximal position for enhanced structural strength.

23. A method for maintaining surgical drainage tube patency using gravity-independent mechanical transport, comprising:
providing a surgical drainage tube having a proximal end with dedicated entry point for screw-coil insertion and separately preserved outflow lumen, and a distal end with drainage apertures;
inserting said distal end into a patient body cavity requiring surgical drainage;
inserting a directional screw-coil assembly through said dedicated entry point into said tube, said screw-coil assembly comprising helical blades with defined handedness and structural struts angled toward said distal end at angles less than 90 degrees relative to a central axis;
rotating said screw-coil assembly in a rotational direction matched to said helical blade handedness to create mechanical transport of fluid and clotted material from said distal end toward said proximal end; and
automatically controlling said rotation through programmable intermittent operation cycles to maintain tube patency throughout a post-operative period.

24. The method of claim 23, wherein said rotating comprises operating at lower speeds of 100-1000 RPM during normal drainage conditions to provide gentle mechanical transport and increasing to higher speeds of 10,000-20,000 RPM when clot accumulation is detected to provide morcellization of clots.

25. The method of claim 23, further comprising diverting drainage material at a Y-connector at said proximal end, wherein drainage material flowing from said distal end toward said proximal end is naturally diverted to said separately preserved outflow lumen through coaxial alignment and soft-angled transition geometry, while said screw-coil assembly operates through a dedicated insertion port.
